(19) United States
(12) Patent Application Publication
(10) Pub. No.: US 2025/0032085 A1
(43) Pub. Date: Jan. 30, 2025

(54) METHOD AND APPARATUS FOR CONE BEAM BREAST CT AND ULTRASOUND HYBRID IMAGING

(71) Applicant: Koning Corporation, Norcross, GA (US)

(72) Inventors: Ruola NING, Atlanta, GA (US); Shachua LIU, Atlanta, GA (US)

(73) Assignee: Koning Corporation, Norcross, GA (US)

(21) Appl. No.: 18/914,210

(22) Filed: Oct. 13, 2024

Related U.S. Application Data
(63) Continuation of application No. PCT/US2023/018728, filed on Apr. 14, 2023.

(51) Int. Cl. A61B 8/08; G06T 7/00

(52) U.S. Cl. CPC A61B 8/0825; G06T 7/00; G06T 2207/30068

(57) ABSTRACT

A cone beam breast computer tomographic imaging system includes a subsystem for ultrasound scanning such that the same system can produce a cone beam breast computer tomographic image, an ultrasound scan image, and a hybrid image that is the fusion of a cone beam breast computer tomographic image and an ultrasound scan image.

### CROSS-REFERENCE TO RELATED APPLICATIONS

The present application is a continuation of PCT patent application PCT/US2023/018728 filed on Apr. 14, 2023, which claims the benefit of provisional patent applications OMNIBUS DISCLOSURE, set forth in an application for Letters Patent of the United States already filed on Apr. 14, 2022 as U.S. Provisional Application No. 63/331,153, and FIXTURING AND SUPPORT FOR MEDICAL IMAGING, set forth in an application for Letters Patent of the United States already filed on Aug. 26, 2022 as U.S. Provisional Application No. 63/401,475, and ERGONOMIC IMPROVEMENTS IN CONE BEAM BREAST COMPUTED TOMOGRAPHY, set forth in an application for Letters Patent of the United States already filed on Aug. 26, 2022 as U.S. Provisional Application No. 63/401,493, and STATIONARY DETAIL IMAGING IN CONE BEAM BREAST COMPUTED TOMOGRAPHY, set forth in an application for Letters Patent of the United States already filed on Aug. 26, 2022 as U.S. Provisional Application No. 63/401,513, and CONE BEAM BREAST COMPUTED TOMOGRAPHY WITH PATIENT SUPPORT SUBSYSTEM, set forth in an application for Letters Patent of the United States already filed on Aug. 26, 2022 as U.S. Provisional Application No. 63/401,546, and, CONE BEAM BREAST COMPUTED TOMOGRAPHY WITH PIVOTAL GANTRY SUBSYSTEM, set forth in an application for Letters Patent of the United States already filed on Aug. 26, 2022 as U.S. Provisional Application No. 63/401,548, and ULTRASONIC HYBRID IMAGING IN CONE BEAM BREAST COMPUTED TOMOGRAPHY, set forth in an application for Letters Patent of the United States already filed on Dec. 6, 2022 as U.S. Provisional Application No. 63/430,571, and CALCIFICATION DETECTION SYSTEMS, METHODS AND APPARATUS IN CONE BEAM BREAST COMPUTED TOMOGRAPHY, set forth in an application for Letters Patent of the United States already filed on Apr. 13, 2023 as U.S. Provisional Application No. 63,459,250, the disclosures of all of which are herewith incorporated by reference in their entireties.

### FIELD OF THE INVENTION

The present invention relates to the field of cone beam tomographic imaging, and in particular to image hybridization of ultrasound and x-ray cone beam breast tomographic imaging.

### SUMMARY

According to the National Cancer Institute, one out of eight women will be diagnosed with breast cancer in her lifetime. And while a reduction in mortality from breast cancer is evident in published reports, each year 40,000 women will die of the disease.

The optimal breast imaging technique detects tumor masses when they are small, preferably less than 10 mm in diameter. It is reported that 93% of women with mammographically detected invasive breast carcinoma 1-10 mm have a 16-year survival rate. In addition, as the diameter of the tumor at detection decreases, the probability of metastasis declines sharply. If a breast tumor is detected when it is 10 mm or less, the probability of metastasis will be equal to 7.31%. If a 4 mm carcinoma is detected, the metastatic probability will be decreased by more than a factor of 10, to 0.617%. These statistics make clear the importance of early detection, while tumors are still small.

Previously, mammography, which on average can detect cancers about 12 mm in size, was the most effective tool for the early detection of breast cancer. Mammography has relatively low sensitivity to small breast cancers (under several millimeters). Specificity and the positive predictive value of mammography remain limited owing to structure and tissue overlap. The limited sensitivity and specificity in breast cancer detection of mammography are due to its poor contrast detectability, which is common for all types of projection imaging techniques (projection imaging can only have up to 10% contrast detectability), and mammography initially detects only 65-70% of breast cancers. The sensitivity of mammography is further reduced to as low as 30% in the dense breast.

Digital mammography (DM) was developed to try to overcome the limitations inherent in screen-film mammography (SFM) by providing improved contrast resolution and digital image processing; however, a large-scale clinical trial, the Digital Mammographic Imaging Screening Trial (DMIST), showed that the rates of false positives for DM and SFM were the same.

The relatively low specificity of mammography leads to biopsy for indeterminate cases, despite the disadvantages of added cost and the stress it imposes on patients. Nearly 80% of the over one million breast biopsies performed annually in the U.S. to evaluate suspicious mammographic findings are benign, burdening patients with excessive anxiety and the healthcare system with tremendous cost. There is a need for more accurate characterization of breast lesions in order to reduce the biopsy rate and the false-positive rate of pre-biopsy mammograms.

In conventional mammography breast imaging is preceded by insertion of a patient's breast into a fixturing apparatus that significantly compresses breast tissue in a direction transverse to a breast longitudinal axis. Patients widely report physical and psychological discomfort related to this compression, and studies have shown that this discomfort is a contributing factor to low rates of screening and diagnostic mammography among patients generally and, in particular, among some ethnic and cultural populations.

Moreover, the breast compression associated with mammography can result in a displacement of breast tissue that impedes the later localization, for biopsy and lumpectomy procedures, of features such as lesions and calcifications.

To address the limitations indicated above, one of the inventors has previously developed a Cone Beam Breast Computed Tomography (CBBCT). Briefly, the major features of CBBCT include a horizontal, ergonomically designed patient table with a modular insert to optimize coverage of the uncompressed breast, including the chest wall; wide openings (1 m) on each side of the patient table for easy access to the breast for positioning and potentially good access for imaging-guided biopsy and other procedures without significantly changing the basic platform; and slip-ring technology that facilitates efficient dynamic contrast imaging studies and angiogenesis imaging in the future.

The results of phantom studies indicate that CBBCT can achieve a spatial resolution up to about 2.8 lp/mm, allowing detection of a 2 mm carcinoma and microcalcifications about 0.2 mm in size for an average size breast (about 13 cm in diameter at the chest wall) with a total dose of about 5 mGy. This dose is less than that of a single mammography exam, assuming two views are required for each breast.

The image quality of CBBCT is excellent for visualizing breast tissues, breast tumors and calcifications, and coverage of the breast, including the chest wall region, is at least equivalent to mammography. Visualization of major blood vessels is very good without using a contrast agent. Accordingly, CBBCT offers significant improvement in detecting and biopsying suspected lesions in a patient.

Additional improvements in CBBCT imaging offer the potential to expand on these benefits. In particular, the addition of hybrid imaging techniques to CBBCT x-ray, as disclosed herewith, allow significantly enhanced image resolution and improve the detection of cancers, especially for a dense breast. For example, the inventors have understood that x-ray CBBCT imaging will be substantially improved by a system in which x-ray CBBCT and ultrasound scanning are combined in a hybrid system to offer significantly increased benefits leading to improved diagnostic effectiveness.

Previously, the possibility of hybridization of scanning techniques has been explored in a variety of imaging technologies. For example, positron emission tomography (PET) has been employed in combination with Magnetic Resonance Imaging (MRI) and with conventional x-ray computed tomography (CT); Single Photon Emission Computed Tomography (SPECT) has been employed in combination with MRI and with CT; Ultrasound Imaging has also been employed in combination with MRI and CT, where separate data sets are acquired in discrete imaging systems and subsequently combined; and MRI has been employed in combination with conventional CT.

It is also known to practice a computed tomography ultrasound imaging, in which a breast to be imaged is disposed within a tank of fluid that serves as an impedance coupling between the ultrasonic transducers and the breast tissue. This requirement for fluid contact presents a number of practical challenges including maintaining hygienic conditions and overcoming potential patient ergonomic concerns.

It will be appreciated that breast tissue is inherently elastic and deformable, and has varying durometer both within a given breast and from breast to breast. Accordingly, any motion of a patient between scans will result in displacement of tissues and rearrangement of peripheral and surface regions of the breast as well as, in some cases, internal feature locations.

The present invention offers the ability to scan a breast while maintaining that breast substantially immobile with respect to the reference frame of the imaging system. In certain embodiments, a single x-ray CBBCT scan can be performed, immediately followed by a single ultrasound imaging scan; in other embodiments, the order of single scans will be reversed. In still other embodiments, multiple x-ray CBBCT scans and/or multiple ultrasound scans can be performed sequentially; and, in still other embodiments, data acquisition sub-cycles of an x-ray CBBCT scan and ultrasound imaging scans can be interleaved.

Depending on the requirements of a particular patient or procedure, the individual scans, or portions of scans, will employ a variety of technical modalities. For example, ultrasound scans will include, in respective portions, reflective ultrasound scans, transmission ultrasound scans, density images and sound speed images, all in conventional and/or tomographic format. In still other embodiments of the invention, x-ray CBBCT and ultrasound imaging scans can be performed concurrently by, for example, rotating the x-ray gantry and ultrasound subsystem rotating apparatus synchronously, with the ultrasound transducer being disposed to admit pulses generally transverse to the longitudinal axis of the x-ray beam and thus with the ultrasound transducer being disposed substantially outside of the x-ray beam.

In certain embodiments of the invention, the data from the x-ray scans and the ultrasound scans will be hybridized after completion of a full rotation of the gantry. In other embodiments of the invention, data from the x-ray scans and the ultrasound scans will be hybridized in real time as each partial scan is performed.

In still other embodiments of the invention, the differential performance advantages of two or more hybrid scanning modalities are employed to complement one another. Thus, for example, in one exemplary method according to principles of the invention, an x-ray CBBCT scan is conducted to produce a data set characterizing a subject breast. X-ray CBBCT imaging is particularly sensitive in the detection of, for example, calcifications within breast tissue. However, in certain presentations, it may be less sensitive to the characterization of benign tumors than ultrasound imaging.

In certain implementations, ultrasound imaging will offer less information about calcifications than x-ray CBBCT imaging, but will provide higher sensitivity for the detection of the presence of cysts in dense breast tissue. Sound speed modalities of ultrasound imaging (and particularly in reflective mode operation) will, in certain embodiments and applications, be especially effective for detecting cancerous tissue at the fat-glandular interface (FGI). Further, ultrasound imaging can provide data as to breast density that complements the high-resolution breast density distribution available from x-ray CBBCT processes.

X-ray CBBCT can provide an attenuation coefficient matrix for individual voxels that characterize the breast in terms of attenuation coefficient for localized tissue regions. Moreover, in certain applications CBBCT imaging will produce an ultra-isotropic image of high spatial resolution. By appropriate combination and analysis of respective parameters, regional stiffness of the breast tissue can be characterized with particular diagnostic effect in dense breast tissue. According to certain aspects of the invention, these benefits are achieved at low x-ray dosage and without the uncomfortable and image-distorting mechanisms of mammographic breast compression. Accordingly, the techniques, apparatus, systems and methods of the present invention are effective in providing high resolution multimodal characterization of the breast in a way that addresses the particular weaknesses of mammographic imaging.

In certain embodiments of the invention, hybrid scanning will be optional. Accordingly, where a preliminary x-ray CBBCT scan identifies a potential region of interest with respect to, e.g., a possible tumor, a subsequent ultrasonic scan will be performed to further characterize features within the region of interest. In certain circumstances, a standard ultrasonic scanning trajectory is employed. In other embodiments of the invention, a specialized and/or customized scanning trajectory will be employed.

Thus, according to the methods and apparatus of some embodiments of the invention, a particular scanning trajectory will be identified, either by an operator, or by an automatic function of the system. In certain embodiments of the invention, the identified specialized scanning trajectory will be one of a plurality of predefined scanning trajectories prepared in advance, as, for example, during design and/or manufacturing of the hybrid scanning system. In such an embodiment, the specialized scanning trajectory will be characterized by a set of data defining parameters of the scan such as, for example, transducer pathway, instantaneous transducer velocity, spatial orientation of an ultrasonic transducer, etc. The characterizing data set will, for example, be stored in a computer memory device of the system and available where beneficial to guide the various motions of the ultrasonic transducer.

In other embodiments of the invention, a specialized scanning trajectory will be prepared during or after the time interval corresponding to the x-ray CBBCT scan, such that the particular parameters and characteristics of the trajectory defined will be identified based on the data produced by the x-ray CBBCT scan. In certain embodiments of the invention, the specialized scanning trajectory will include predefined trajectory elements and customized trajectory elements combined to produce a resulting overall trajectory.

The following description is provided to enable any person skilled in the art to make and use the disclosed inventions and sets forth the best modes presently contemplated by the inventors of carrying out their inventions. In the following description, for purposes of explanation, numerous specific details are set forth in order to provide a thorough understanding of the present invention. It will be apparent to one skilled in the art, however, that the present invention may be practiced without these specific details. In other instances, well-known structures and devices are shown in block diagram form in order to avoid unnecessarily obscuring the substance disclosed. These and other advantages and features of the invention will be more readily understood in relation to the following detailed description of the invention, which is provided in conjunction with the accompanying drawings.

It should be noted that, while the various figures show respective aspects of the invention, no one figure is intended to show the entire invention. Rather, the figures together illustrate the invention in its various aspects and principles. As such, it should not be presumed that any particular figure is exclusively related to a discrete aspect or species of the invention. To the contrary, one of skill in the art will appreciate that the figures taken together reflect various embodiments exemplifying the totality of the invention.

Correspondingly, referenced throughout the specification to “one embodiment” or “an embodiment” means that a particular feature, structure, or characteristic described in connection with the embodiment is included in at least one embodiment of the present invention. Thus, the appearance of phrases such as, for example, “in one embodiment”, “in an embodiment” or “in certain embodiments” in various places throughout the specification are not necessarily all referring to the same embodiment. Furthermore, the particular features, structures, or characteristics may be combined in any suitable manner in one or more embodiments.

### BRIEF DESCRIPTION OF THE DRAWINGS

FIG. 1 shows in schematic perspective view, a portion of an exemplary CBBCT imaging system;

FIG. 2 shows, in schematic perspective view, a portion of an exemplary CBBCT imaging system including an ultrasound imaging subsystem prepared according to principles of the invention;

FIG. 3A shows, in schematic cutaway perspective view, a portion of an exemplary CBBCT imaging system including an ultrasound imaging subsystem prepared according to principles of the invention;

FIG. 3B shows, in schematic perspective view, a portion of an exemplary CBBCT imaging system including further aspects and configurations of an ultrasound imaging subsystem prepared according to principles of the invention;

FIG. 3C shows, in schematic perspective view, a portion of an exemplary CBBCT imaging system including further aspects and modes of operation of an ultrasound imaging subsystem prepared according to principles of the invention;

FIG. 4A shows, in schematic perspective view, a portion of an exemplary CBBCT imaging system including certain details of an ultrasound imaging subsystem prepared according to principles of the invention;

FIG. 4B shows, in schematic block diagram form, a portion of an exemplary CBBCT imaging system including certain details of an ultrasound imaging subsystem prepared according to principles of the invention;

FIG. 5 shows, in schematic perspective view, a portion of an exemplary CBBCT imaging system including further aspects and modes of operation of an ultrasound imaging subsystem including transmission ultrasound apparatus prepared according to principles of the invention;

FIG. 6A shows, in schematic perspective view, a portion of a further exemplary CBBCT imaging system including an ultrasound imaging subsystem prepared according to principles of the invention;

FIG. 6B shows, in schematic perspective view, a portion of a further exemplary CBBCT imaging system including further aspects and configurations of an ultrasound imaging subsystem prepared according to principles of the invention;

FIG. 7A shows, in schematic cutaway perspective view, a portion of a further exemplary CBBCT imaging system including an ultrasound imaging subsystem prepared according to principles of the invention;

FIG. 7B shows, in schematic perspective view, a portion of a further exemplary CBBCT imaging system including further aspects and configurations of an ultrasound imaging subsystem prepared according to principles of the invention;

FIG. 8A shows, in schematic perspective view, a portion of a further exemplary CBBCT imaging system including an ultrasound imaging subsystem prepared according to principles of the invention;

FIG. 8B shows, in schematic perspective view, a portion of a further exemplary CBBCT imaging system including an ultrasound imaging subsystem prepared according to principles of the invention;

FIG. 9 shows, in schematic perspective view, further aspects of a hybrid CBBCT imaging system including a multi-ring ultrasound imaging subsystem; and

FIG. 10 shows, in schematic block diagram form, exemplary aspects of processing systems and methods according to principles of the invention.

### DETAILED DESCRIPTION

The following description is provided to enable any person skilled in the art to make and use the disclosed inventions and sets forth the best modes presently contemplated by the inventors for carrying out their inventions. In the following description, for purposes of explanation, numerous specific details are set forth in order to provide a thorough understanding of the present invention. It will be apparent, however, to one skilled in the art that the present invention may be practiced without these specific details.

It should be noted that while any of the embodiments described for exemplary purposes below will identify specific elements and combinations of elements, these examples are not intended to be determinative. Rather, discrete elements will, in appropriate circumstances, be combined into integral elements and/or assemblies. Further, the present disclosure of aspects and features of particular elements described herewith as integral, should be understood to convey also the disclosure of individual elements and assemblies providing the same characteristics and/or functionality.

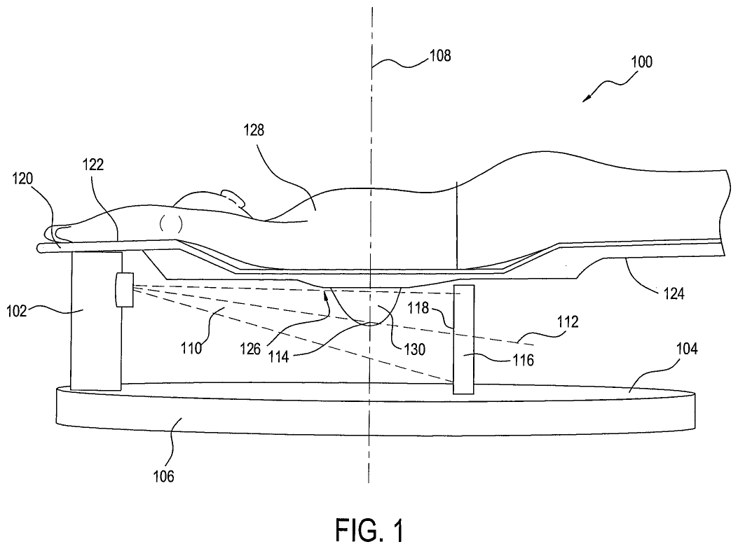

FIG. 1 shows, in schematic perspective view, a portion of an exemplary CBBCT imaging system 100. The system 100 includes an x-ray source 102. The x-ray source 102 is mounted on an upper surface 104 of a rotating gantry 106. The rotating gantry 106 is supported by a bearing, and arranged for rotation about an axis of rotation 108.

The x-ray source 102 is configured to emit a beam of x-rays 110. The beam of x-rays 110 defines a beam longitudinal axis 112 that, in the illustrated embodiment, intersects (at 114) the axis of rotation 108. In certain embodiments of the invention, beam 110 is configured as a cone beam. In certain configurations, a cross-section of the beam 110 taken transverse to the longitudinal axis 112 defines a disk of substantially uniform x-ray intensity with a substantially circular perimeter.

In other configurations within the scope of the invention, a cross-section of the beam 110 taken transverse to the longitudinal axis 112 defines a region of substantially uniform x-ray intensity with a substantially circular perimeter save for a portion of the disc outwardly of a chord of said circular perimeter. As will be appreciated on consideration of the further disclosure below, in certain embodiments, the chord will be disposed in generally parallel spaced relation to a lower surface of a patient table.

Accordingly, in certain configurations, a cross-section of the beam 110 taken transverse to the longitudinal axis 112 defines a truncated disk of substantially uniform x-ray intensity with a substantially truncated circular perimeter (i.e., a perimeter that is circular except for a horizontal chord of the circle at its upper periphery). This configuration optimizes imaging of the breast while minimizing irradiation of chest wall tissue above the breast. It is implemented, in certain embodiments, by the placement of an x-ray-opaque collimating plate across a portion of an otherwise circular cross-section beam generated by the x-ray source.

In still further configurations within the scope of the invention, a cross-section of the beam 110 taken transverse to the longitudinal axis 112 defines a region of substantially uniform x-ray intensity with a polygonal perimeter, where the polygonal perimeter will, in respective embodiments and configurations, include any of a triangular perimeter, a rectangular perimeter, a pentagonal perimeter, a hexagonal perimeter, a perimeter of any higher geometric shape, or a perimeter having any arbitrary curve or combination of line segments and curves according to the demands of a particular application. Moreover, it will be appreciated that any of the cross-sectional configurations described above may define a beam having a nonuniform intensity including, without limitation an intensity that falls to zero in a region, or certain regions, of the cross-section.

An x-ray detector 116 is also mounted on the upper surface 104 of the rotating gantry 106. In one exemplary embodiment, the x-ray detector 116 includes a flat panel detector having a generally planar receiving surface 118. Receiving surface 118 is disposed generally transverse to longitudinal axis 112 and on the opposite side of axis of rotation 108 from the x-ray source 102. It will be appreciated by one of skill in the art that the configuration described is merely exemplary of many possible arrangements in which the x-ray source, the x-ray detector, and any other component of the system, maybe supported from above, from a side, or in any other way appropriate to achieving the desired function, and that the shape and configuration of the gantry, and of the x-ray detector, will likewise assume any useful form in respective embodiments of the invention.

Accordingly, in certain embodiments of the invention, the x-ray detector 116 will include an x-ray detector having a curved receiving surface 118. In other embodiments of the invention, the x-ray detector 116 will include an x-ray detector having a flexible receiving surface 118. In certain embodiments of the invention, a curvature of the x-ray detector surface will be variable, and in certain embodiments of the invention, a curvature or other configuration of the receiving surface 118 will be adjustable in anticipation of, or during the course of a particular x-ray CBBCT scan.

In other embodiments of the invention, a receiving surface 118 of the x-ray detector 116 will terminate in close proximity to a lower surface region of the patient table. In certain embodiments of the invention, a receiving surface 118 of the x-ray detector 116 will have an upper edge within at least about 10 mm of a lower surface region of the patient table. In further embodiments of the invention, a receiving surface 118 of the x-ray detector 116 will have an upper edge disposed within at least about 5 mm of a lower surface region of the patient table. In still further embodiments of the invention, a receiving surface 118 of the x-ray detector 116 will have an upper edge disposed within at least about 3 mm of a lower surface region of the patient table.

Rotation of the gantry 106 about axis of rotation 108 during operation of the imaging system 100 will result in the receiving surface 118 following a transit path about axis of rotation 108. In a typical configuration, the transit path will include at least a portion of a circle disposed transverse to, and centered at, axis of rotation 108. It should be noted, however, that other transit paths are considered to be within the scope of the invention, and to be disclosed herewith.

In certain embodiments of the invention, one or both of the x-ray source 102 and the x-ray detector 116 are arranged so that their respective positions on the upper surface 104 of gantry 106 are adjustable. For example, the x-ray source 102 and the x-ray detector 116 may be adjustable in a radial direction with respect to axis of rotation 108, in a circumferential direction with respect to axis of rotation 108, in a direction towards or away from gantry surface 104, or in any other manner deemed beneficial by a designer or user of a particular apparatus embodying the invention.

A patient table 120 includes an upper surface 122 and a lower surface 124. An aperture 126 communicates between the upper surface 122 and lower surface 124 of the table. The upper surface 122 is arranged to support a patient 128, typically with the patient lying prone on surface 122, as illustrated. In this arrangement, a breast 130 of the patient is disposed pendant from the patient's chest wall downwardly through aperture 126.

In operation, the gantry rotates about axis of rotation 108, carrying x-ray source 102 and x-ray detector 116 in transit in a path around the patient's breast. During this transit, x-ray image data is captured by operation of the x-ray detector 116 in conjunction with corresponding interface electronics and computer systems. The x-ray image data corresponds to a plurality of x-ray images taken at respective angular locations about axis of rotation 108. Taken together, the x-ray image data, or a subset of the same, is processed to provide information about an internal state of the breast.

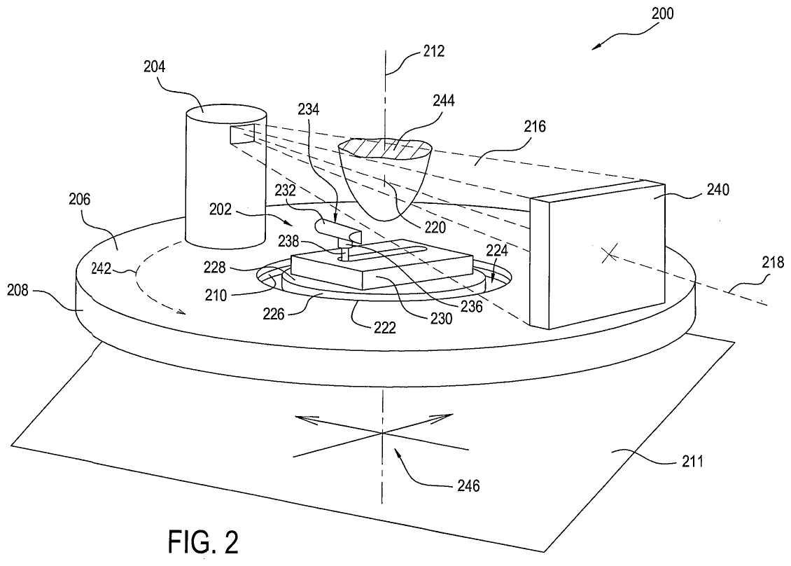

FIG. 2 shows in schematic perspective view, a CBBCT imaging system 200, including an ultrasound imaging subsystem 202, prepared according to principles of the invention. Similar to system 100, described above, system 200 includes an x-ray source 204. The x-ray source 204 is mounted on an upper surface 206 of a rotating gantry 208. The rotating gantry 208 is supported by a bearing 210, and arranged for rotation about an axis of rotation 212. The bearing 210 is, in turn, supported by a structural member 211 of the imaging system 200 or, alternately, by a floor.

The x-ray source 204 is configured to emit a beam of x-rays 216. The beam of x-rays 216 defines a beam longitudinal axis 218 that, in the illustrated embodiment, intersects (at 220) the axis of rotation 212.

In the illustrated embodiment, the upper surface 206 of the rotating gantry 208 includes an internal circumferential edge 222. The internal circumferential edge 222 defines an aperture 224 through the upper surface 206 of the gantry 208.

In certain embodiments of the invention, the rotating gantry 208 includes a slip-ring for communicating power and/or electronic signals and/or optical signals on and off of the gantry. In certain embodiments of the invention, the slip ring includes an aperture disposed generally coaxially with the aperture 224 of the gantry.

In other embodiments of the invention, information and control signals are communicated on and off of the gantry through wireless modulated electromagnetic radiation signals including, without limitation, any of radiofrequency, microwave, and/or optical frequency electromagnetic radiation.

In the illustrated embodiment, rotary apparatus 226 is disposed within aperture 224. In certain aspects and embodiments of the invention, the rotary apparatus 226 is supported by the structural member 211 of the imaging system 200 or, alternately, by the floor.

As illustrated, the exemplary rotary apparatus 226 has an upper surface 228. The rotary apparatus 226 permits rotation about axis of rotation 212 of the upper surface 228 with respect to the floor and, optionally, with respect to the longitudinal axis 218 of the x-ray beam 216 (as well as the gantry 208, the x-ray source 204, and the room frame of reference 246).

Accordingly, in certain embodiments of the invention, upper surface 228 of the rotary apparatus 226 will rotate independently of upper surface 206 of the gantry 208. In further embodiments and aspects of the invention, upper surface 228 of the rotary apparatus 226 will remain stationary with respect to the room while the upper surface 206 of the gantry 208 rotates with respect to the room. In still other embodiments of the invention, rotation of the upper surface 228 of rotary apparatus 226 will be synchronized to rotation of the upper surface 206 of the gantry 208 (hereinafter, co-rotation), and in still other embodiments and aspects of the invention, upper surface 228 of the rotary apparatus 226 will counter-rotate with respect to the upper surface 206 of the gantry 208 and the room 246. In still other embodiments and aspects of the invention, upper surface 228 of the rotary apparatus 226 will co-rotate with respect to the upper surface 206 of the gantry 208, but out of phase with upper surface 206 of the gantry 208 (hereinafter, differential rotation) with respect to the room 246.

In certain embodiments of the invention, differential rotation and/or co-rotation of the upper surface 228 with respect to upper surface 206 will be controlled using, for example, a rotary drive including elements such as, for example, an electric motor such as, e.g., a squirrel cage motor, a pancake motor or a capacitor start motor, a stepper motor, a DC electric or other servo motor along with a feedback device such as, for example, a resolver, an optical encoder, a magnetic encoder or a resistive encoder, a hydraulic motor, a pneumatic motor, a spring motor, a piezoelectric motor, a gear train including, for example, one or more pinion gears or one or more worm gears, a ratchet drive mechanism, a chain and sprocket mechanism, a belt and sheave mechanism, and/or a timing belt and timing pulley mechanism, including any combination of the foregoing and any other appropriate device and/or mechanism known, or that becomes available to, one of skill in the art.

In certain embodiments of the invention, differential rotation and/or co-rotation of the upper surface 228 with respect to upper surface 206 will be controlled using output signals from a system digital computer and/or a system digital controller. In other aspects and embodiments of the invention, differential rotation and/or co-rotation of the upper surface 228 with respect to upper surface 206 will be controlled using dedicated hardware such as, for example, embedded specialized motion control hardware including, for example, motion control integrated circuits, power amplifiers, feedback elements including, for example, limit switches, Hall effect sensors, optical sensors, resolvers, optical encoders, resistive encoders, and the like, any of which will be employed in implementing generalized motion control and/or phase locked loop control.

In still other embodiments and aspects of the invention, differential rotation and/or co-rotation of the upper surface 228 with respect to upper surface 206 is controlled using a mechanical coupling between the upper surface 228 and the upper surface 206 where, in certain embodiments, the mechanical coupling will be controllable subject to manual and/or automatic configuration and control and where, in certain embodiments, the mechanical coupling includes one or more of a solenoid controlled latch, a rotary motor control latch, a pneumatically controlled latch, a hydraulically controlled latch, a manually operated latch, a gear train, a drive chain, a belt or timing belt, a mechanical, hydraulic or electrical clutch, any of the foregoing in any desirable combination (and in a manner that would be made clear by the present disclosure along with a minimum of experimentation to one of skill in the art), and any other appropriate mechanism known, or that becomes available, to one of skill in the art.

The ultrasound imaging subsystem 202 has a base portion 230 including a translation apparatus and a first ultrasound transducer assembly 232. In the exemplary illustration of system 200, base portion 230 is coupled to and supported by upper surface 228 of rotary apparatus 226.

In the illustrated example, first ultrasound transducer assembly 232 has a first breast contact surface region 234, a pivotal apparatus 236 and a support column 238.

In certain embodiments of the invention, the translation apparatus includes a first translation portion. The first translation portion is operatively coupled between the base portion 230 and the first ultrasound transducer assembly 232.

As previously discussed, during CBBCT imaging, gantry 208 is rotated about axis of rotation 212, carrying x-ray source 204 and an imager 240, which are disposed on and supported by upper surface 206, or otherwise coupled to and supported by gantry 208, along a transit path 242 about axis of rotation 212 while a breast 244 being imaged remains substantially stationary with respect to a room frame of reference 246.

It will be appreciated by one of skill in the art that, depending on the material, arrangement and configuration of the first ultrasound transducer assembly 232, it will be preferable in some circumstances that the x-ray beam 216 not pass through the first ultrasound transducer assembly 232 during CBBCT imaging. Rather, the first ultrasound transducer assembly will be displaced out of the path of the x-ray beam 216 during CBBCT imaging. Accordingly, as illustrated in FIG. 2, system 200 is arranged with the ultrasound imaging subsystem 202 in a storage configuration wherein the first ultrasound transducer assembly 232 is disposed in a retracted state.

One of skill in the art will appreciate that, in alternative arrangements and embodiments, the ultrasound imaging subsystem 202 will remain in a deployed configuration (as described below) during CBBCT scanning, rather than in the storage configuration described above. In such embodiments, the x-ray beam will pass parallel to breast contact surface region 234 such that the first ultrasound transducer assembly 232 overlaps the resulting breast image in a minimal fashion or not at all. In other embodiments of the invention, image features related to the first ultrasound transducer assembly 232 will be deleted from the resulting image by signal processing techniques including, for example and without limitation, data filtering techniques, as known in the art.

In the illustrated storage configuration of FIG. 2, x-ray beam 216 passes through breast 244 and on to imager 240 without substantially impinging on the first ultrasound transducer assembly 232. This condition is maintained, in certain embodiments of the invention, by rotation of upper surface 228 in coordination with the rotation of upper surface 206 throughout the CBBCT scan. One of skill in the art will appreciate that this coordination will, in respective embodiments, be maintained throughout the length of transit path 242, whether that transit path traverses 360° through room frame of reference 246, 180°, or any other angular extent according to the requirements of a particular CBBCT imaging protocol.

It will also be appreciated, in light of the foregoing discussion, that this coordination between the surface regions 228 and 206 will be maintained, in various embodiments, through mechanical, electronic, or analog or digital servo control or other control means.

In other embodiments, (as shown, for example, in FIG. 2) the storage configuration of the first ultrasound transducer assembly 232 will be such that the x-ray beam 216 will clear (i.e., not impinge on) the first ultrasound transducer assembly 232, notwithstanding rotation of surface 206. Therefore, surface region 228 can be allowed to remain stationary with respect to the room frame of reference 246 during rotation of surface 206.

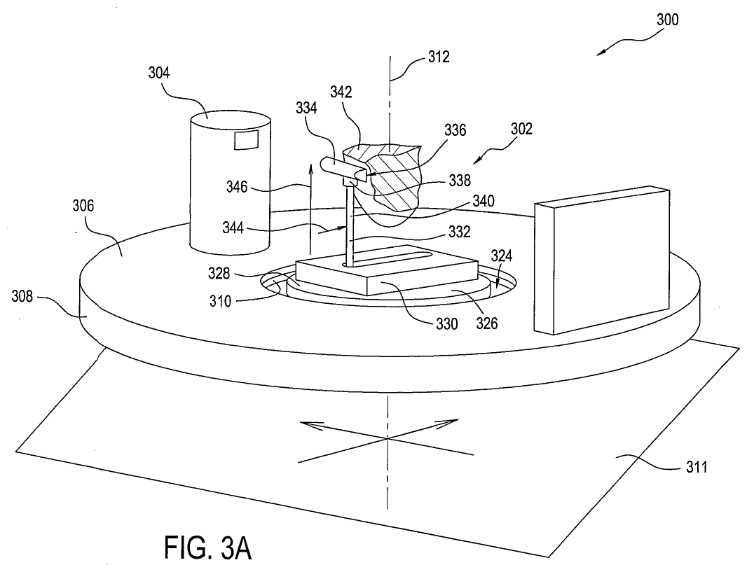

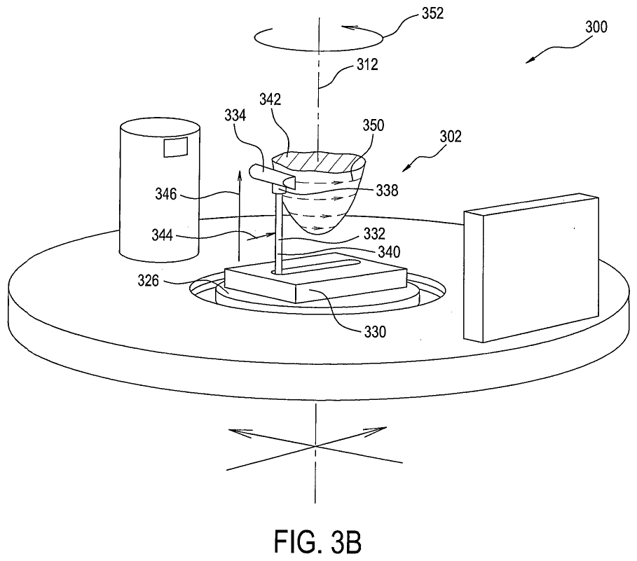

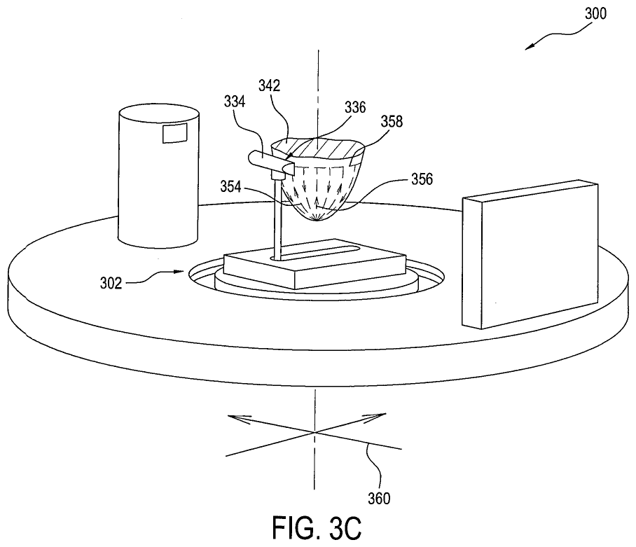

FIG. 3A shows in schematic perspective cutaway view, further aspects of a CBBCT imaging system 300, including an ultrasound imaging subsystem 302, prepared according to principles of the invention. Similar to systems 100 and 200, described above, system 300 includes an x-ray source 304. The x-ray source 304 is mounted on an upper surface 306 of a rotating gantry 308. The rotating gantry 308 is supported by a bearing 310, and arranged for rotation about an axis of rotation 312. The bearing 310 is, in turn, supported by a structural member 311 of the imaging system 300 or, alternately, by a floor.

In the illustrated embodiment, a rotary apparatus 326 is disposed within an aperture 324. In certain aspects and embodiments of the invention, the rotary apparatus 326 is supported by a structural member 311 of the imaging system 300 or, alternately, by the floor.

Consistent with the description of system 200 above, ultrasound imaging subsystem 302 has a base portion 330 including a translation apparatus and a first ultrasound transducer assembly 332. In the exemplary illustration, base portion 330 is coupled to and supported by upper surface 328 of rotary apparatus 326.

In the illustrated example, the ultrasound transducer assembly 332 has an ultrasound transducer portion 334 with a first breast contact surface region 336, a pivotal apparatus 338 and a support column 340.

In contrast to the configuration shown in FIG. 2, where the first ultrasound transducer assembly 232 is shown in a storage configuration, the first ultrasound transducer assembly 332 is shown in a deployed configuration, where the breast contact surface region 336 is disposed in contact with a corresponding surface region of a breast 342 to be imaged.

Accordingly, as compared with the location of the first ultrasound transducer assembly 232 shown in FIG. 2, first ultrasound transducer assembly 332 is shown disposed inwardly in dimension 344 and upwardly in dimension 346, both taken with respect to a reference frame of base portion 330 of the ultrasound subsystem 302. This serves to place the first breast contact surface region 336 into contact with a corresponding surface region of the breast 342. As will be further discussed below, during operation of the ultrasound imaging subsystem 302 the position and orientation of the ultrasound transducer portion 334 are controlled so that the first breast contact surface region 336 follows a controlled transit path across an external surface region of the breast 342.

FIG. 3B shows in schematic perspective view, further aspects and configurations of CBBCT imaging system 300, including ultrasound imaging subsystem 302. As noted above, the ultrasound imaging subsystem 302 includes a rotary apparatus 326 supporting a base portion 330 and a first ultrasound transducer assembly 332.

In the exemplary arrangement illustrated, during operation, the ultrasound transducer portion 334 of the first ultrasound transducer assembly 332 is adapted and controlled to follow a generally circumferential spiral-helical transit path 350 circumferentially over an external surface region of a breast 342 being imaged. As will be apparent in light of the foregoing, this transit path is achieved in the illustrated embodiment by a rotation 352 of rotary apparatus 326 about an axis of rotation 312, coordinated with horizontal and vertical motions of support column 340 in dimensions 344 and 346 respectively, and coordinated with pitch, yaw and roll motions of a pivotal apparatus 338, all in the frame of reference of the base portion 330.

FIG. 3C shows an alternative arrangement in which a CBBCT scanning system 300 includes an ultrasound subsystem 302 with a first ultrasound transducer portion 334. In contrast to the configuration of FIG. 3B, a breast surface contact region 336 of the first ultrasound transducer portion 334 follows a transit path 354 that is not spiral-helical over an external surface region of the breast 342 being imaged but, rather, the transit path traverses the breast surface radially/longitudinally 356, with respective horizontal circumferential increments 358 between longitudinal sweeps 356.

A practitioner of ordinary skill in the art will, in light of the present disclosure and with a minimum of experimentation, arrive at a wide variety of other transit paths, including paths having linear and nonlinear portions, and paths including combinations of any of the foregoing that serve to effectively capture ultrasonic data that will be hybridized with data collected by the x-ray CBBCT portion of the CBBCT imaging system 300. In certain embodiments these transit paths will be hardwired into the system (i.e., by actual electromechanical construction or by pre-storage of firmware or software), and multiple hardwired paths may be alternatively or combinationally available and selectable in respective embodiments. In other embodiments of the invention, transit paths will be programmable and reprogrammable, and in certain embodiments of the invention a unique transit path will be prepared according to the characteristics, parameters and requirements of a particular patient.

It should be noted that, in contrast to a method effecting a combination of data from a pre-existing CBBCT system and a separate pre-existing ultrasound system (such as, for example, a conventional ultrasound system, a robotic/automatic ultrasound system, or an ultrasound computed tomography system) the system, apparatus and methods of the present invention offer unique and novel advantages. In particular, the combined hybrid system described herewith allows the imaging of the breast with two or more modalities without physical/spatial displacement of the patient. Accordingly, problems of registration and coordination of data sets, as well as spatial rearrangement of the breast tissue between data acquisition cycles are reduced or eliminated.

It will be appreciated that breast tissue is inherently elastic and deformable, and has varying durometer both within a given breast and from breast to breast. Accordingly, any motion of a patient between scans will result in displacement of tissues and rearrangement of peripheral and surface regions of breast as well as, in some cases, internal feature locations.

The present invention offers the ability to scan a breast while maintaining that breast substantially immobile with respect to the reference frame 360 of the imaging system. In certain embodiments, a single x-ray CBBCT scan can be performed, immediately followed by a single ultrasound imaging scan; in other embodiments, the order of single scans will be reversed. In still other embodiments, multiple x-ray CBBCT scans and/or multiple ultrasound scans can be performed sequentially; and, in still other embodiments, data acquisition cycles of an x-ray CBBCT scan and ultrasound imaging scans can be interleaved.

Depending on the requirements of a particular patient or procedure, the individual scans, or portions of scans, will employ a variety of technical modalities. For example, ultrasound scans will include, in respective portions, reflective ultrasound scans, transmission ultrasound scans, density images and sound speed images, all in conventional and/or tomographic format. In still other embodiments of the invention, x-ray CBBCT and ultrasound imaging scans can be performed concurrently by, for example, rotating the x-ray gantry and ultrasound subsystem rotating apparatus synchronously, with the ultrasound transducer being disposed to admit pulses generally transverse to the longitudinal axis of the x-ray beam and thus with the ultrasound transducer being disposed substantially outside of the x-ray beam.

In certain embodiments of the invention, the data from the x-ray scans and the ultrasonic scans will be hybridized after completion of a full rotation of the gantry. In other embodiments of the invention, data from the x-ray scans and the ultrasound scans will be hybridized in real time as each partial scan is performed.

In still other embodiments of the invention, the differential performance advantages of two or more hybrid scanning modalities are employed to complement one another. Thus, for example, in one exemplary method according to principles of the invention, an x-ray CBBCT scan is conducted to produce a data set characterizing a subject breast. X-ray CBBCT imaging is particularly sensitive in the detection of, for example, calcifications within breast tissue. However, in certain presentations, it may be less sensitive to the characterization of benign tumors than ultrasound imaging.

In certain implementations, ultrasound imaging will offer less information about calcifications than x-ray CBBCT imaging, but will provide higher sensitivity for the detection of the presence of cysts in dense breast tissue. Sound speed modalities of ultrasound imaging (and particularly in reflective mode operation) will, in certain embodiments and applications, be especially effective for detecting cancerous tissue at the fat-glandular interface (FGI). Further, ultrasound imaging can provide data as to breast density that complements the high-resolution breast density distribution available from x-ray CBBCT processes.

X-ray CBBCT can provide an attenuation coefficient matrix for individual voxels that characterizes the breast in terms of attenuation coefficient for localized tissue regions. Moreover, in certain applications CBBCT imaging will produce an ultra-isotropic image of high spatial resolution. By appropriate combination and analysis of respective parameters, regional stiffness of the breast tissue can be characterized with particular diagnostic effect in dense breast tissue. According to certain aspects of the invention, these benefits are achieved at low x-ray dosage and without the uncomfortable and image-distorting mechanisms of mammographic breast compression. Accordingly, the techniques, apparatus, systems and methods of the present invention are effective in providing high resolution multimodal characterization of the breast in a way that addresses the particular weaknesses of mammographic imaging.

In certain embodiments of the invention, hybrid scanning will be optional. Accordingly, where a preliminary x-ray CBBCT scan identifies a potential region of interest with respect to, e.g., a possible tumor, a subsequent ultrasonic scan will be performed to further characterize features within the region of interest. In certain circumstances, a standard ultrasonic scanning trajectory such as e.g., trajectory 350 of FIG. 3B, or trajectory 354 of FIG. 3C will be employed. In other embodiments of the invention, however, a specialized or customized scanning trajectory will be employed.

Thus, in according to the methods and apparatus of some embodiments of the invention, a particular scanning trajectory will be identified, either by an operator, or by an automatic function of the system. In certain embodiments of the invention, the identified specialized scanning trajectory will be one of a plurality of predefined scanning trajectories prepared in advance, as, for example, during manufacture of the hybrid scanning system. In such an embodiment, the specialized scanning trajectory will be characterized by a set of data defining parameters of the scan such as, for example, transducer pathway, instantaneous transducer velocity, spatial orientation of an ultrasonic transducer, etc. The characterizing data set will, for example, be stored in a computer memory device of the system and available where beneficial to guide the various motions of the ultrasonic transducer.

In other embodiments of the invention, a specialized scanning trajectory will be prepared during or after the time interval corresponding to the x-ray CBBCT scan, such that the particular parameters and characteristics of the trajectory defined will be identified based on the data produced by the x-ray CBBCT scan.

In a still further embodiment of the invention, a specialized scanning trajectory is prepared by medical or technical personnel operating the system. In such an embodiment of the invention, the medical or technical personnel define the scanning trajectory of the ultrasonic imaging subsystem based on available information including data produced by the x-ray CBBCT scan, as well as supplementary information such as external features and appearances of the breast being scanned, two-dimensional x-ray images, computed tomography images, MRI images, PET images, SPECT images, etc.

In certain embodiments of the invention, the technical or medical personnel operate a user interface of the hybrid CBBCT imaging system. In certain embodiments of the invention, the user interface includes a touchscreen interface that allows selecting of a particular region and/or definition of a particular path in the context of a displayed CBBCT three-dimensional image. In certain embodiments of the invention, the ultrasound scanning subsystem is operated after definition of the path using the touchscreen interface.

In other embodiments of the invention, the user interface of the hybrid CBBCT imaging system includes, for example, a plurality of push buttons, a plurality of knobs, a sensor equipped glove system, a joystick, or other user interface modality, or combination of the same, to allow definition of the ultrasonic scanning trajectory in real time as the ultrasonic image is produced.

While CBBCT system generates 3D reconstruction attenuation coefficient of breast, tumors and calcifications, CBBCT images can guide ultrasound scan to further characterize the tumors, specially for the dense breast.

hybrid CBBCT system can also guide biophsy and minimal invasive treatment to further improve accuracy, reduce procedure time and reduce the dose required by using CBBCT alone. CBBCT is able to provide true 3D high resolution imaging of the breast, the location of the breast lesions. However, CBBCT is not optimal for real time imaging. Ultrasound is optimal for real time imaging while it is not optimal for 3D high resolution mapping and can not detect calcifications. Hybrid systems can provide both the high resolution 3D location of the breast lesions and real time imaging guidance.

Accordingly, an operator such as, e.g., medical or technical personnel, is able to view the x-ray CBBCT image produced by a preliminary CBBCT scan and then operate the user interface to guide ultrasonic transducer of the ultrasonic imaging subsystem over a customized trajectory to secure additional information about a region of interest. In various embodiments and applications of the invention the resulting scanning trajectory will follow any possible trajectory over surface of the breast including substantially linear trajectories, substantially curved trajectories, oscillatory and/or recursive trajectories, and any other trajectory that produces a desirable improvement in image quality. In certain embodiments of the invention, data from the ultrasonic imaging system is hybridized with data from the CBBCT imaging system to produce an image of improved resolution or stability or other desirable characteristic so as to further characterize the region of interest of the breast tissue.

In certain embodiments of the invention, hardware or software based image processing of the CBBCT and/or ultrasonic image data will be applied to further clarify the image by, for example, adding false color to represent tissue characteristics, identifying transitions and borders within the tissue, filtering extraneous information such as intervening features of the breast or the system, or otherwise improving the presentation of the data available.

It will be apparent to one of skill in the art that, whereas other systems may be available that hybridize the results of x-ray computed tomography imaging with ultrasonics, the present systems and methods offer unique advantages inasmuch as a plurality of imaging modalities will be applied while the breast being imaged remains substantially immobile within the system. Accordingly, wide variety of registration and image stitching system demands, including demands that offer the opportunity for the introduction of image artifacts, will be avoided by the present system.

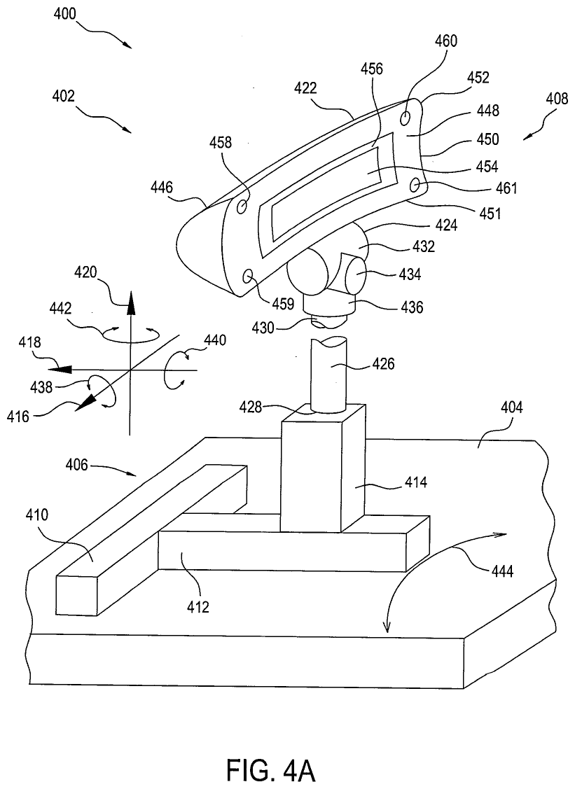

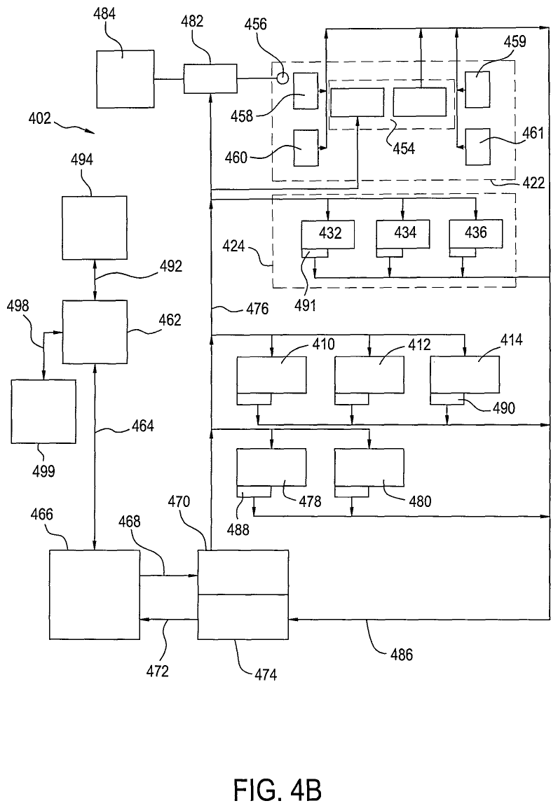

FIG. 4A shows, in schematic perspective view, portions and additional details of a CBBCT scanning system 400 that includes an ultrasound imaging subsystem 402. The ultrasound imaging subsystem 402 has a base portion 404. The base portion 404 is mounted on, for example, a rotary apparatus like rotary apparatus 226 of FIG. 2. The ultrasound imaging subsystem 402 includes a translation portion 406 and a first ultrasound transducer assembly 408, forming, in effect, a robot subsystem.

The illustrated exemplary translation portion 406 includes a first circumferential actuator 410, a second radial actuator 412, and a third vertical actuator 414. Circumferential actuator 410 is arranged and configured to move the ultrasound transducer assembly 408 in a first circumferential dimension/degree of freedom 416, as taken, e.g., with respect to the axis of rotation 212 shown in FIG. 2. Second radial actuator 412 is arranged and configured to move the ultrasound transducer assembly 408 in a second radial dimension/degree of freedom 418, and third vertical actuator 414 is arranged and configured to move the ultrasound transducer assembly 408 in a third vertical dimension/degree of freedom 420.

In certain embodiments of the invention, one or more of the circumferential actuator 410, the radial actuator 412 and the vertical actuator 414, will be implemented as a linear actuator. As will be appreciated by one of skill in the art, the herewith-described linear actuators (and any of the linear actuators referenced herewith) can be implemented with a wide variety of actuators available in the art. For example, in certain embodiments, the linear actuator will include one or more of a pneumatic cylinder; a pneumatic bladder; a pneumatic bellows; a hydraulic cylinder; a hydraulic bladder; a hydraulic bellows; an electrical solenoid; a thermoelectric actuator; a shape memory alloy actuator; a piezoelectric actuator; a linear stepping motor; a rotary actuator, such as an electric motor (or any of the actuators above), along with, e.g., a rack and pinion apparatus, a rack and worm gear apparatus, an Acme screw and Acme nut; a ballscrew apparatus; transverse complementary ramps; a scissors linkage mechanism, including, for example, a scissors linkage mechanism linkage operated by a lead screw, a sarrus linkage mechanism; a cable and pulley arrangement; a timing belt and timing pulley arrangement; a ratchet and pawl driver; a compressive spring; a tension spring; a torsion spring; a coil spring; a reducer including, without limitation, a spur gear reducer, a worm gear reducer, a harmonic reducer, an assembly of leaf springs; a spring including a plurality of Belleville washers; a shock absorbing coupling; a flexible coupling; a universal joint; and/or a flexible shaft; as well as any of a wide variety of manual actuators such as, for example, a handcrank and/or a ratchet lever; or any other linear actuator currently known, or that becomes known in the art, that is suited to the requirements of a particular application and to providing the requisite extension function.

In the illustration of FIG. 4A, the exemplary ultrasound transducer assembly 408 includes an ultrasound transducer portion 422, a pivotal apparatus 424 and a support column 426. Support column 426 has a first end 428. First end 428 is coupled to a corresponding portion of the third vertical actuator 414. Support column 426 has a second end 430. Second end 430 is coupled to a corresponding portion of the pivotal apparatus 424. The support column 426 serves to couple the third vertical actuator 414 to the pivotal apparatus 424 and, through the pivotal apparatus 424, to the ultrasound transducer portion 422.

The exemplary pivotal apparatus 424 includes first 432, second 434, and third 436 rotary actuators that allow controlled pivotal motion of the ultrasound transducer portion 422 in pitch 438, roll 440, and yaw 442 degrees of freedom respectively. As will be appreciated in light of the foregoing disclosure, motion of a rotary apparatus (e.g., 226 of FIG. 2, or of a CBBCT gantry, e.g. 208) will provide a further rotary degree of freedom 444 to the range of motion of the ultrasound transducer portion 422.

The ultrasound transducer portion 422 will include a body or shell portion 446 and a breast contacting surface region 448. In certain embodiments of the invention, breast contacting surface region 448 will embody a spatial curvature (visible, e.g., at peripheral edge 450, including peripheral edge portion 451) adapted to enhance effective contact between the breast contacting surface region 448 and a corresponding external surface region of the breast to be imaged. In certain embodiments of the invention peripheral edge 450 will include rounded corners, e.g., 452 that will serve to minimize the possibility of snagging the breast tissue during ultrasound scanning.

In addition, in certain embodiments of the invention, breast contacting surface region 448 will include a material of appropriate durometer and elastic resilience that likewise serves to improve contact with a corresponding breast surface region, as well as to enhance patient comfort. Accordingly, in certain embodiments, breast contact surface region 448 will include a material such as, for example, a synthetic polymer material or synthetic elastomer material having a relatively high lubricity. In certain embodiments, a foam polymer material will be included.

In certain embodiments of the invention, the ultrasonic transducer portion 422 will include one or more ultrasonic transducers, e.g., 454. In certain embodiments of the invention, the one or more ultrasonic transducers, e.g. 454, will include a discrete ultrasonic actuator/transmitter and a discrete ultrasonic sensor/receiver. In other embodiments of the invention, a single transducer device will serve both as an actuator and sensor, serving to transmit and receive respectively ultrasonic signals into and out of the breast tissue.

In certain embodiments of the invention, the ultrasonic transducer portion 422 will include one or more lubricant ports, e.g., 456. Lubricant ports 456 will, in certain embodiments of the invention dispense, and/or receive a lubricant material during operation of the ultrasonic transducer subsystem 402. As will be understood by one of skill in the art, the lubricant material will serve to lubricate motion of the breast contact surface region 448 in relation to the corresponding surface region of the breast, as well as to provide impedance transformation between the ultrasonic transducer(s) 454 and the tissue of the breast being imaged.

In certain embodiments of the invention, and as illustrated, the ultrasonic transducer portion 422 will include one or more feedback sensors, e.g., 458, 459, 460, 461. Feedback sensors 458, 459, 460, 461 will sense, for example, proximity of the breast interface surface region 448 to the corresponding surface region of the breast to be imaged, pressure between the breast interface surface region 448 and the corresponding surface region of the breast to be imaged, velocity of the breast interface surface region 448 across the corresponding surface region of the breast to be imaged, friction between the breast interface surface region 448 and the corresponding surface region of the breast to be imaged, presence and/or sufficiency of lubricant material between the breast interface surface region 448 and the corresponding surface region of the breast to be imaged, or any other parameter deemed to be useful as a control/feedback parameter of the ultrasound imaging subsystem 402, or of value in a clinical evaluation of the breast to the image, or of the patient.

FIG. 4B shows, in schematic block diagram form, certain features, components, elements and relationships corresponding to ultrasound imaging subsystem 402, as illustrated and described in relation to FIG. 4A. Accordingly, and for exemplary purposes only, a supervisory computer system 462 of a CBBCT hybrid imaging system is operatively coupled 464 to, e.g., an embedded microcontroller subsystem 466. The microcontroller subsystem 466 will, in certain embodiments of the invention, include a dedicated microcontroller as well as memory elements, communication buses, and input/output devices as known in the art.

The microcontroller subsystem 466 is operatively coupled 468 to an output subsystem module 470, the output subsystem module including, e.g., operational amplifiers, output amplifiers and motor controller devices such as, for example, one or more of Servo control devices, stepper motor control devices, linear amplifiers and switching amplifiers, power semiconductors and optical isolators, ultrasonic signal generator elements and ultrasonic signal amplification elements.

The microcontroller subsystem 466 is also operatively coupled 472 to an input subsystem module 474. The input subsystem module 474 includes, e.g., signal conditioning circuits and devices including, for example, one or more of operational amplifiers, signal preamplifiers, signal amplifiers, dedicated digital signal processors, semiconductors and optical isolators, ultrasonic signal receiver elements and ultrasonic signal conditioning elements as well as, in certain embodiments, ultrasonic signal interpretation elements.

The output subsystem module 470 is operatively coupled, directly or via a bus structure 476, to e.g., a rotary actuator 478 for a CBBCT gantry (such as gantry 208 of FIG. 2); a rotary actuator 480 for a rotary apparatus for an ultrasound imaging subsystem (such as rotary apparatus 226 of FIG. 2); linear actuators 410, 412 and 414 of translation portion 406, rotary actuators 432, 434 and 436 of pivotal apparatus 424 and ultrasonic transducer, e.g., 454, and a lubricant pump 482 that, for example, draws lubricant from a lubricant reservoir 484 and supplies the lubricant to a lubricant port 456 of the ultrasonic transducer portion 422.

In the exemplary configuration illustrated, the input subsystem module 474 is operatively coupled, directly or via a bus structure 486, to, e.g., ultrasonic transducer 454, any of sensors e.g., 458, 459, 460, 461 as described above, and respective feedback elements such as, e.g., feedback element 488 of rotary actuator 478, feedback element 490 of linear actuator 414, and feedback element 491 of rotary actuator 432 etc. One of skill in the art will appreciate that any of the actuators illustrated will, in certain embodiments of the invention, include a dedicated feedback element (as illustrated) for automatic feedback control of the same.

It will be appreciated by one of skill in the art that the feedback element will be any feedback element technically appropriate to the respective application. Thus, the feedback element will be, for example and without limitation, an optical encoder, a resolver, a magnetic encoder, a resistive encoder, a Hall effect sensor, and/or any other sensor or apparatus adapted to provide positional, speed, temperature or other information that is useful and/or effective in the control of the respective actuator or apparatus.

It should be further noted that, while the ultrasonic system and sensors of the present exemplary embodiment are indicated as being coupled through the exemplary input and output subsystem modules 470, 474, in alternative embodiments, discrete subsystems will be provided for the generation, receipt and interpretation of signals related to these components and elements. Such discrete subsystems will, in certain embodiments, be directly coupled to the microcontroller subsystem 466 and/or to the supervisory computer system 462.

It will also be understood that, in certain embodiments of the invention, the supervisory computer system 462 will be operatively coupled 492 to a user interface apparatus 494. The user interface apparatus will include any of various interface devices, as known in the art, including, keyboards, microphones, joysticks, pushbuttons, sliders, instrumented gloves, etc. Reference is made, for example, to the user interface referenced above in relation to selecting and creating ultrasound transducer trajectories, but it will be understood that the user interface will also be employed in any appropriate aspect of system control. In addition, in certain embodiments of the invention, the supervisory computers 462 will be operatively coupled 498 to the x-ray CBBCT system 499.

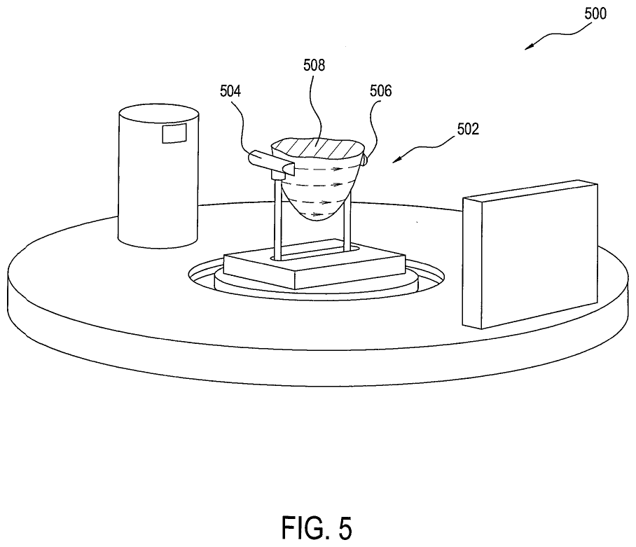

FIG. 5 shows in schematic perspective view, a hybrid CBBCT imaging system 500, including an ultrasound imaging subsystem 502, prepared according to principles of the invention. Ultrasound imaging system 502 can be compared to ultrasound imaging system 302 (illustrated and discussed above) which is shown as a reflective ultrasound system. Reflective ultrasound imaging system 302 has a single ultrasound transducer portion, e.g., 334, that serves as an ultrasound transmitter/receiver. In contrast, ultrasound imaging system 502 illustrates a transmission ultrasound imaging system with separate transmitter and receiver elements.

While it will be appreciated that, in certain embodiments, a reflective ultrasound system will employ a plurality of transducers, it will also be possible to prepare a transmission ultrasound system as shown, for example, in ultrasound imaging system 502.

Ultrasound imaging system 502 includes a first ultrasound transmitter 504, and a second ultrasound receiver 506. In the illustrated embodiment, ultrasound transmitter 504 is disposed diametrically opposite ultrasound receiver 506 with respect to a breast 508 being image. Accordingly, ultrasound signals emitted by ultrasound transmitter 504 are modulated by passage through the tissue of the breast 508 and thereafter received at ultrasound receiver 506. Ultrasound receiver 506 produces a modulated signal that is then interpreted to provide information regarding an internal state of the breast.

It will be understood by one of skill in the art that a variety of other arrangements of ultrasound transmitters and receivers will be employed in respective embodiments of the invention. Accordingly, in corresponding embodiments of the invention, reflection ultrasound apparatus and methods will be employed, as will transmission ultrasound apparatus and methods, and as will combinations of the same.

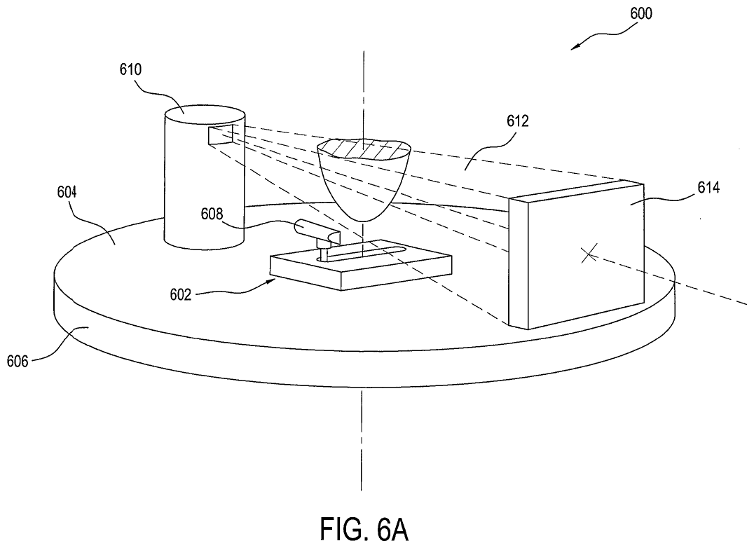

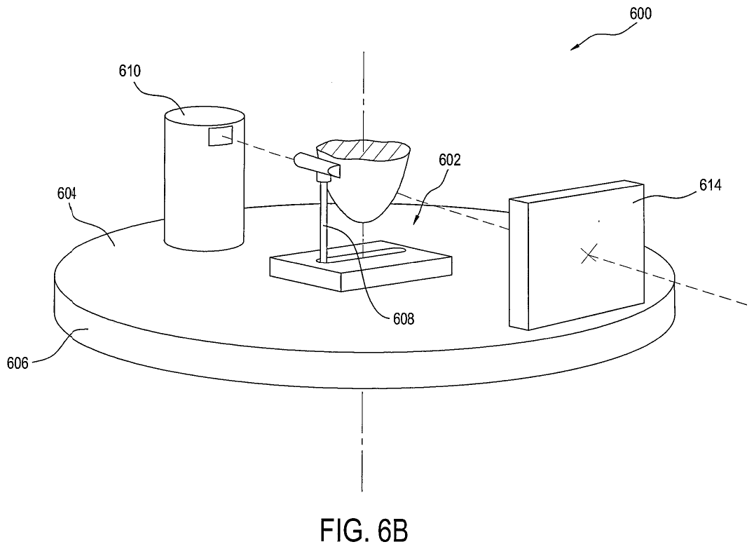

FIGS. 6A and 6B show in schematic perspective view, a hybrid CBBCT imaging system 600, including an ultrasound imaging subsystem 602. In contrast to the imaging systems shown and described above, ultrasound imaging subsystem 602 is substantially fixedly coupled to an upper surface region 604 of a CBBCT gantry 606. Consequently, an ultrasonic transducer assembly 608 rotates synchronously with CBBCT gantry 606 whenever the gantry is rotating. In certain embodiments of the invention, the ultrasound imaging subsystem 602 is detachable and the gantry, and can be removed and reinstalled according to the requirements of a particular patient and/or procedure.

Rotation of the gantry 606 provides the spatial displacement of scanning equipment required for both x-ray CBBCT and ultrasound scanning. In certain embodiments of the invention, an x-ray CBBCT scan is performed first, followed by an ultrasound scan. In other embodiments of the invention, an ultrasound scan is performed first, followed by an x-ray CBBCT scan. In still further embodiments of the invention, the collection of x-ray image data is chronologically interleaved with the collection of ultrasound data, and in still further embodiments of the invention x-ray data is collected concurrently with ultrasound data. While CBBCT system generates 3D reconstruction attenuation coefficient of breast, tumors and calcifications, CBBCT images can guide ultrasound scan to further characterize the tumors, specially for dense breast.

Accordingly, in one aspect of a method according to the invention, a first rotation of the gantry 606 occurs during a first time interval. During the first time interval, an x-ray source 610 produces an x-ray beam 612 received by x-ray detector 614. During the first time interval the ultrasound imaging subsystem 602 is disposed in a storage configuration, as shown in FIG. 6A, and is inactive.

Thereafter, during a second time interval, the x-ray source 610 is inactive and the ultrasound transducer assembly transitions from the first storage configuration to a second deployed configuration as shown in FIG. 6B.

Thereafter, during a third time interval, the x-ray source, remains inactive and the gantry 606 rotates a second time. During the third time interval, the ultrasound transducer assembly 608 traverses all or a portion of an ultrasound scanning trajectory such as, e.g., a circumferential spiral-helical scanning trajectory 350, as shown in FIG. 300.

In certain embodiments, the gantry rotation during the first time interval is a full gantry rotation. In other embodiments of the invention, the gantry rotation during the first time interval is a partial gantry rotation. In certain embodiments of the invention, the gantry rotation during the first time interval traverses at least about 360º. In other embodiments of the invention, the gantry rotation during the first time interval traverses at least about 180° plus the width of the x-ray cone beam. In still further exemplary embodiments of the invention, the gantry rotation during the first time interval traverses at least about 1°; at least about 5°; at least about 10°; at least about 20°; at least about 45°; at least about 90°; at least about 180°; at least about 270°; at least about 720°; or any other rotation deemed desirable or useful in relation to the requirements of a particular patient or modality of operation.

In a still further embodiment of the invention, alternating x-ray and ultrasound slices are captured. Accordingly, during a first time interval, an x-ray pulse is emitted by x-ray source 610 and captured by x-ray detector 614. Thereafter, during a second time interval the ultrasound transducer assembly 608 traverses a first portion of an ultrasonic scanning trajectory such as, for example, a single longitudinal portion 356 of transit path 354 shown in FIG. 3. Thereafter, during a third time interval the gantry 606 rotates a finite increment such as, e.g., 1°, 2°, or 5°, etc. Thereafter, during a fourth time interval, an x-ray pulse is emitted by x-ray source 610 and captured by x-ray detector 614. Thereafter, during a fifth time interval, the ultrasound transducer assembly 608 traverses a first portion of an ultrasonic scanning trajectory such as, for example, a further longitudinal portion like portion 356 of transit path 354 shown in FIG. 3.

It will be appreciated that the sequence of events described in the preceding paragraph will be repeated as many times as required to complete a desired hybrid scan. It will also be understood that, in certain embodiments of the invention, the ultrasound transducer assembly 608 will transition between a storage configuration and a deployed configuration between each x-ray pulse cycle. In other embodiments of the invention, the ultrasound transducer assembly 608 will remain deployed during an x-ray pulse cycle. It should be noted that in alternative embodiments of the invention, the ultrasound subsystem is removable, and may installed into the CBBCT system when ultrasound scanning is required and/or desirable. Accordingly, in certain embodiments of the invention, the ultrasound system is removed during CBBCT scanning is present in the CBBCT system when CBBCT scanning is not underway.

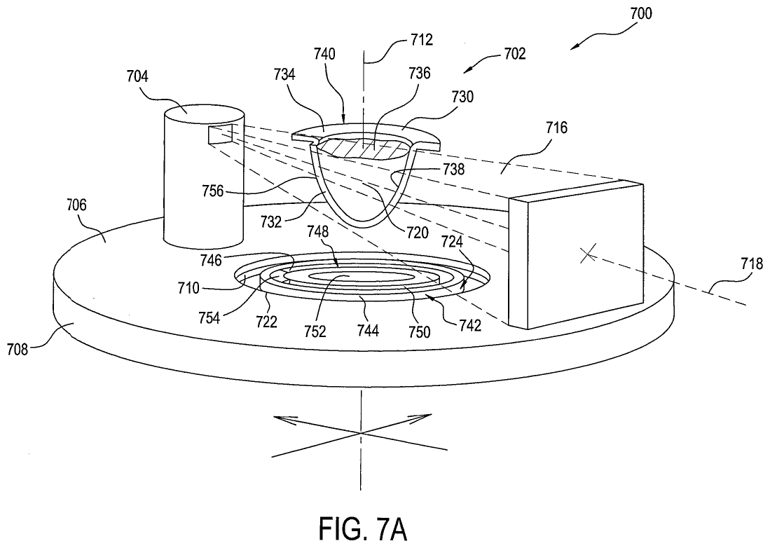

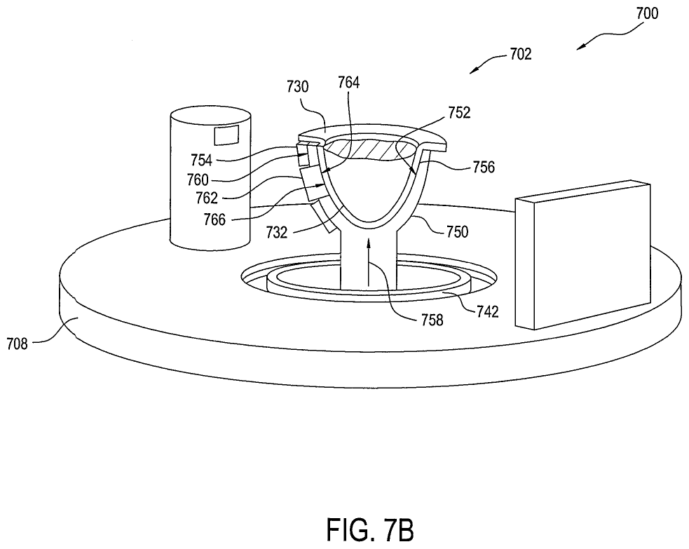

FIGS. 7A and 7B show further aspects of the invention in exemplary embodiments. In these figures, the breast to be imaged is supported and stabilized by a breast stabilizer unit. During a first time interval, x-ray CBBCT imaging is applied to secure a tomographic data set characterizing the breast. During a second time interval, the breast is scanned with, for example, ultrasound technology to secure additional tomographic or other imaging of the breast.

In the illustrated embodiment, an ultrasound transducer assembly is maintained in a storage configuration (as shown in FIG. 7A) during the first time interval, and is moved into a deployed configuration in contact with the breast stabilization unit (as shown in FIG. 7B) during the second time interval. During ultrasound scanning, ultrasonic energy is directed through the breast stabilizer unit to reach the breast to be imaged. Accordingly, one of skill in the art will appreciate that in the embodiments exemplified by FIGS. 7A and 7B, the breast stabilizer unit will include materials optimally selected for both x-ray transparency and satisfactory ultrasonic impedance matching between the ultrasonic transducers and the tissue of the breast to be imaged.

In certain embodiments of the invention, the ultrasound transducer assembly will include a circumferential sliding interface with the breast stabilizer unit. In other embodiments of the invention, the ultrasound transducer assembly will include a plurality of ultrasound transducers arranged and configured to secure effective imaging of the breast without rotary motion of the ultrasound transducer assembly.

In light of the foregoing, FIG. 7A shows in schematic cutaway perspective view, a hybrid CBBCT imaging system 700, including an ultrasound imaging subsystem 702.

Similar to systems 100, 200 and 300 described above, system 700 includes an x-ray source 704. The x-ray source 704 is mounted on an upper surface 706 of a rotating gantry 708. The rotating gantry 708 is supported by a bearing 710, and arranged for rotation about an axis of rotation 712. The bearing 710 is, in turn, supported by a structural member of the imaging system 700 or, alternately, by a floor.

The x-ray source 704 is configured to emit a beam of x-rays 716. The beam of x-rays 716 defines a beam longitudinal axis 718 that, in the illustrated embodiment, intersects (at 720) the axis of rotation 712.

In the illustrated embodiment, the upper surface 706 of the rotating gantry 708 includes an internal circumferential edge 722. The internal circumferential edge 722 defines an aperture 724 through the upper surface 706 of the gantry 708.

In certain embodiments of the invention, the rotating gantry 708 includes a slip-ring for communicating power and/or electronic signals and/or optical signals on and off of the gantry. In certain embodiments of the invention, the slip ring includes an aperture disposed generally coaxially with the aperture 724 of the gantry.

In other embodiments of the invention, information and control signals are communicated on and off of the gantry through wireless modulated electromagnetic radiation signals including, without limitation, any of radiofrequency, microwave, and/or optical frequency electromagnetic radiation.

In the illustrated embodiment, a breast stabilizer unit 730 is disposed above aperture 724. The illustrated breast stabilizer unit 730 includes a body portion 732 and a flange portion 734. Exemplary of certain embodiments, the breast stabilizer unit 730 is supported by the flange portion 734 at an aperture of a patient interface table (as with, e.g., aperture 126 of patient table 120, shown in FIG. 1). The breast stabilizer unit supports and stabilizes the breast 736 during imaging.

As illustrated, the breast stabilizer unit 730 includes an internal surface region 738 that defines a recess 740 of the breast stabilizer unit. During imaging, the breast 736 is disposed within the recess 740, and an external surface region of the breast 736 is disposed in contact with the internal surface region 738 of the breast stabilizer unit 730.

The various characteristics and benefits of a general breast stabilizer unit are shown and discussed, along with a variety of exemplary embodiments, in co-pending United States non-provisional patent application serial number < . . . >, filed on < . . . >, the disclosure of which is herewith incorporated by reference in its entirety. [K9-01-P1]

In the illustrated embodiment, a rotary apparatus 742 is disposed within the aperture 724 of the gantry 708. The exemplary rotary apparatus 742 illustrated includes a rotary apparatus body portion 744 with a rotary apparatus internal surface region 746 that defines a recess 748 within the rotary apparatus body portion 744 of the rotary apparatus 742.

In FIG. 7A an ultrasound transducer assembly 750 is shown disposed in a storage configuration within recess 748 of the rotary apparatus body portion 744 of the rotary apparatus 742. As will be further discussed below, the ultrasound transducer assembly 750 includes an internal interface surface region 752 and an ultrasonic transducer 754. During operation of the ultrasound imaging subsystem 702, the internal interface surface region 752 is disposed adjacent to and/or in contact with an external surface region 756 of the breast stabilizer unit 730.

FIG. 7B shows in schematic cutaway perspective view, the hybrid CBBCT imaging system 700, with the ultrasound transducer assembly 750 of the ultrasound imaging subsystem 702 in a deployed configuration.

In the deployed configuration, the internal interface surface region 752 of the ultrasound transducer assembly 750 is disposed adjacent to, or in intimate contact with, the external surface region 756 of the body portion 732 of the breast stabilizer unit 730. In embodiments where appreciable distance exists between the external surface region 756 and the internal interface surface region 752, a lubricant material of appropriately selected acoustic impedance may be provided within that space to reduce friction between the two surfaces and to reduce the reflection of ultrasonic energy at the interfaces. In other embodiments of the invention, intimate contact between external surface region 756 and the internal interface surface region 752 is maintained by, e.g., pressure applied to the ultrasound transducer assembly 750 by, e.g., a linear actuator of the rotary apparatus 742 urging the ultrasound transducer assembly 750 in a direction/degree of freedom 758.

In certain embodiments of the invention, the lubricant material may be supplied through pores or orifices and under pressure, so as to provide a sufficient amount and/or continuous flow of lubricant material between the internal interface surface region 752 and the external surface region 756 (i.e., within the interface region).

In certain embodiments of the invention, an ultrasound transducer, e.g., ultrasound transducer 754 will be coupled to an external surface region 760 of the ultrasound transducer assembly 750. In other embodiments of the invention, an ultrasound transducer, e.g., ultrasound transducer 762 will be supported by the ultrasound transducer assembly 750, but the ultrasound transducer 762 will include a transducer surface region 764 disposed in direct contact (and in sliding engagement) with a corresponding surface region 766 of external surface region 756 of the breast stabilizer unit 730.

In certain embodiments of the invention, the rotary apparatus 742 will rotate during ultrasonic imaging, with a corresponding rotation of the ultrasound transducer assembly 750 and, accordingly, the ultrasound transducers e.g., 754, 762. In other embodiments of the invention, the rotary apparatus 742 will be omitted and the ultrasound imaging subsystem 702 will be directly coupled to the CBBCT gantry 708. In such embodiments, rotation of the ultrasound transducer assembly 750 will be achieved by rotation of the entire CBBCT gantry 708. In still further embodiments of the invention, a plurality and/or arrangement of ultrasound transducers will be included in the ultrasound transducer assembly 750 such that ultrasonic imaging is achieved without rotation of the ultrasound transducer assembly 750 (as, for example, by direct imaging and/or by synthetic aperture techniques).

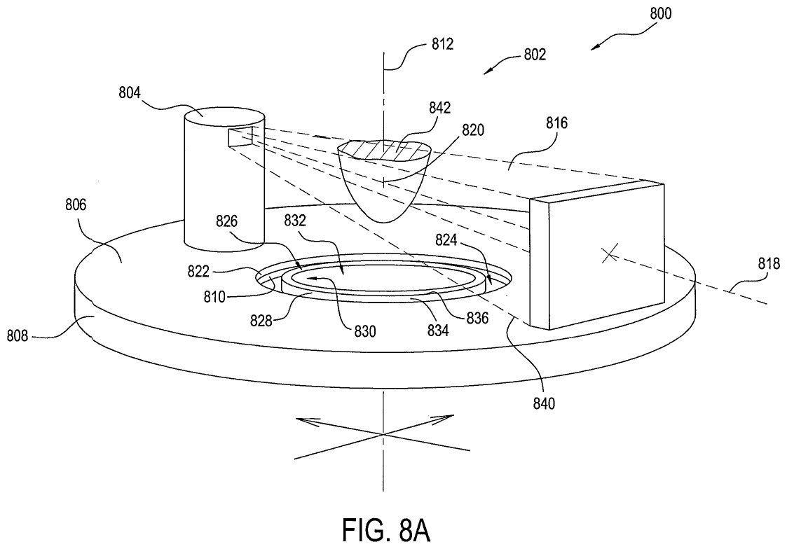

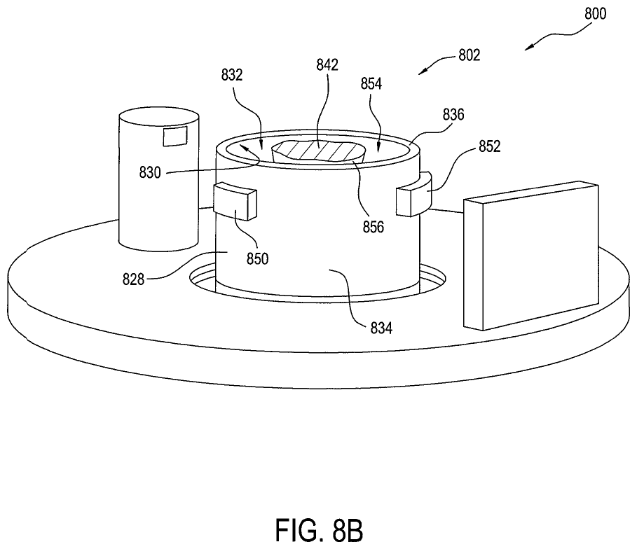

FIGS. 8A and 8B show further aspects of the invention in exemplary embodiments. In these figures, x-ray CBBCT imaging is conducted to characterize a patient breast. Using the same system, ultrasound imaging of the breast is conducted while the breast is immersed in an impedance matching fluid such as, for example and without limitation, water.

In the illustrated embodiment, an ultrasound transducer assembly is maintained in a storage configuration (as shown in FIG. 8A) during a first time interval, and is moved into an imaging configuration (as shown in FIG. 8B) during a second time interval. During the first time interval, x-ray CBBCT imaging is applied to secure a tomographic data set characterizing the breast. During the second time interval, the breast is immersed in the impedance matching fluid and scanned with ultrasound energy to secure ultrasound tomographic (or other ultrasound imaging) of the breast.

In certain embodiments of the invention, the immersed breast will be contained within, and supported by, a breast stabilizer in the nature of the breast stabilizer described above with respect to FIG. 7A. In other embodiments of the invention, the immersed breast will be contained within a flexible membrane of synthetic polymer or other material. In still other embodiments of the invention, the breast will be directly immersed in the impedance matching fluid without any intervening substance.

In light of the foregoing, FIG. 8A shows in schematic perspective view, a hybrid CBBCT imaging system 800, including an ultrasound imaging subsystem 802.

Similar to systems 100, 200, 300 and 700 described above, system 800 includes an x-ray source 804. The x-ray source 804 is mounted on an upper surface 806 of a rotating gantry 808. The rotating gantry 808 is supported by a bearing 810, and arranged for rotation about an axis of rotation 812. The bearing 810 is, in turn, supported by a structural member of the imaging system 800 or, alternately, by a floor.

The x-ray source 804 is configured to emit a beam of x-rays 816. The beam of x-rays 816 defines a beam longitudinal axis 818 that, in the illustrated embodiment, intersects (at 820) the axis of rotation 812.

In the illustrated embodiment, the upper surface region 806 of the rotating gantry 808 includes an internal circumferential edge 822. The internal circumferential edge 822 defines an aperture 824 through the upper surface 806 of the gantry 808.

In certain embodiments of the invention, the rotating gantry 808 includes a slip-ring for communicating power and/or electronic signals and/or optical signals on and off of the gantry. In certain embodiments of the invention, the slip ring includes an aperture disposed generally coaxially with the aperture 824 of the gantry.

In other embodiments of the invention, information and control signals are communicated on and off of the gantry through wireless modulated electromagnetic radiation signals including, without limitation, any of radiofrequency, microwave, and/or optical frequency electromagnetic radiation, or by any other means that are operative and known or discovered in the art.

In the illustrated embodiment, the hybrid CBBCT imaging system 800 includes the ultrasound imaging subsystem 802 disposed within the aperture 824 of the gantry 808. The ultrasound imaging subsystem 802 includes a rotary apparatus 826 with a body portion 828 having an internal surface region 830. Internal surface region 830 defines a recess 832, such that the body portion 828 with recess 832 defines an imaging tank 834.

When disposed in the storage configuration, as shown in FIG. 8A, an upper peripheral surface region 836 of imaging tank 834 is disposed below a lower extremity 840 of x-ray beam 816. Consequently, the presence of the ultrasonic imaging subsystem 802 does not interfere with x-ray CBBCT imaging of the breast 842.

FIG. 8B shows, in schematic perspective view, further aspects of a hybrid CBBCT imaging system 800, including an ultrasound imaging subsystem 802. The ultrasound imaging system 802 is disposed in the imaging configuration, such that the imaging tank 834 is elevated above upper surface region 806 of rotating gantry 808, and the upper peripheral surface region 836 of imaging tank 834 is disposed in proximity to a chest wall of a patient lying prone above the imaging tank and supported on a patient support table (or other apparatus) (not shown).

As illustrated, the breast 842 to be imaged is disposed pendant from the chest wall and within recess 832 of the body portion 828. The body portion 828 includes and/or supports at least one ultrasonic transducer 850. In other embodiments of the invention, the body portion 828 includes and/or supports at least a second ultrasonic transducer 852. In certain embodiments, ultrasonic transducer 850 operates as both an ultrasonic transmitter and an ultrasonic receiver. In other embodiments of the invention, ultrasonic transducer 850 operates as an ultrasonic transmitter and ultrasonic transducer 852 operates concurrently as an ultrasonic receiver.

In certain methods of operation according to principles of the invention, recess 832 of the body portion 828 contains a coupling material 854 disposed between internal surface region 830 and an external surface region 856 of breast 842. In respective embodiments of the invention, the coupling material will, for example and without limitation, include a liquid material, a gel material, a phase change material, a mixture of materials, including for example a picture of materials adapted to cure so as to exhibit a change of physical characteristics such as fluidity and/or durometer, solid material, or any other material deemed to be useful or beneficial in relation to the requirements of a particular patient or procedure.

In certain embodiments of the invention, the rotary apparatus is urged between the first storage configuration and the second imaging configuration by an apparatus including for example, a linear actuator. As will be appreciated by one of skill in the art, the herewith-described linear actuators (and any of the linear actuators referenced herewith) can be implemented with a wide variety of actuators available in the art. For example, in certain embodiments, the linear actuator will include one or more of a pneumatic cylinder; a pneumatic bladder; a pneumatic bellows; a hydraulic cylinder; a hydraulic bladder; a hydraulic bellows; an electrical solenoid; a thermoelectric actuator; a shape memory alloy actuator; a piezoelectric actuator; a linear stepping motor; a rotary actuator, such as an electric motor (or any of the actuators above), along with, e.g., a rack and pinion apparatus, a rack and worm gear apparatus, an Acme screw and Acme nut; a ballscrew apparatus; transverse complementary ramps; a scissors linkage mechanism, including, for example, a scissors linkage mechanism linkage operated by a lead screw, a sarrus linkage mechanism; a cable and pulley arrangement; a timing belt and timing pulley arrangement; a ratchet and pawl driver; a compressive spring; a tension spring; a torsion spring; a coil spring; a reducer including, without limitation, a spur gear reducer, a worm gear reducer, a harmonic reducer, an assembly of leaf springs; a spring including a plurality of Belleville washers; a shock absorbing coupling; a flexible coupling; a universal joint; and/or a flexible shaft; as well as any of a wide variety of manual actuators such as, for example, a handcrank and/or a ratchet lever; or any other linear actuator currently known, or that becomes known in the art, that is suited to the requirements of a particular application and to providing the requisite extension function.

A CBBCT imaging subsystem generates 3D reconstruction image of a breast with isotropic resolution. A tomographic ultrasound subsystem generates 3D ultrasound tomographic image of a breast. The Hybrid CBBCT-Ultrasound system generates multiple correlation 3D images or fusion images to provide complementary imaging information of a breast.

A hybrid CBBCT-Ultrasound system can be used for image-guided biopsy or minimal-invasive treatment. A CBBCT imaging subsystem provides accurate location of a lesion and 3D treatment space coordinate, and an ultrasound subsystem provides real time image of the biopsy needle or treatment tool (RF Poles). The hybrid CBBCT-Ultrasound system can provide the real time imaging guidance for biopsy or treatment by fusing CBBCT image with real time ultrasound image of the biopsy needle.

To fuse CBBCT image and ultrasound image, a few references markers will be imaged in both CBBCT and ultrasound system. In exemplary embodiments of the invention, a marker may be as simple as a vitamin E capsule adhere to the skin of the breast. In other embodiments of the invention, the registration marker will be provided on the breast using an x-ray-translucent or x-ray-opaque ink. In still other embodiments of the invention, and adhesive sticker or bandage with a metallic or other x-ray opaque or translucent material is provided as a registration marker.

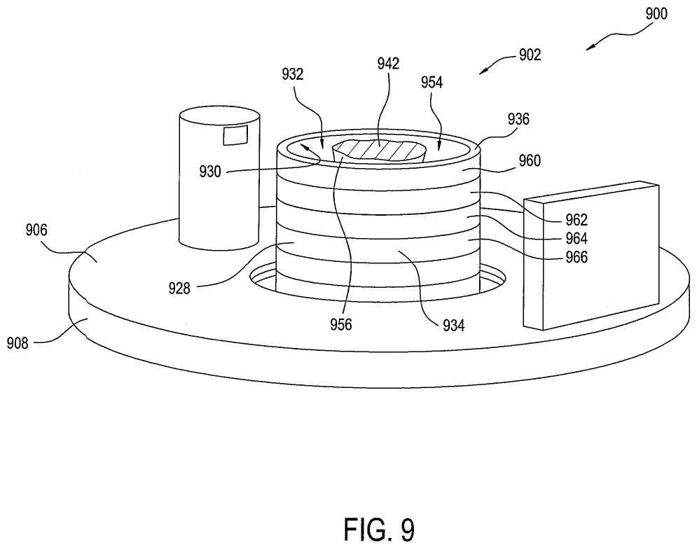

FIG. 9 shows, in schematic perspective view, aspects of a further exemplary hybrid CBBCT imaging system 900, including an ultrasound imaging subsystem 902. The ultrasound imaging system 902 is shown disposed in an imaging configuration, such that an imaging tank 934 is elevated above upper surface region 906 of a rotating gantry 908, and an upper peripheral surface region 936 of imaging tank 934 is disposed in proximity to a chest wall of a patient lying prone above the imaging tank and supported on a patient support table (or other apparatus) (not shown).

As illustrated, the breast 942 to be imaged is disposed pendant from the chest wall and within recess 932 of the body portion 928. The body portion 928 includes and/or supports one or more ring ultrasonic transducer assemblies, e.g., 960, 962, 964, 966. In certain embodiments, each ultrasonic transducer assembly, e.g., 960 operates as both an ultrasonic transmitter and an ultrasonic receiver.

In certain methods of operation according to principles of the invention, recess 932 of the body portion 928 contains a coupling material 954 disposed between internal surface region 930 and an external surface region 956 of breast 942. In respective embodiments of the invention, the coupling material will, for example and without limitation, include a liquid material, a gel material, a phase change material, a mixture of materials, including for example a picture of materials adapted to cure so as to exhibit a change of physical characteristics such as fluidity and/or durometer, solid material, or any other material deemed to be useful or beneficial in relation to the requirements of a particular patient or procedure. As will be appreciated by 1 of skill in the art, the coupling material will serve as impedance matching transformer between a transducer of the dances for assembly, e.g., 960, and the breast 942.

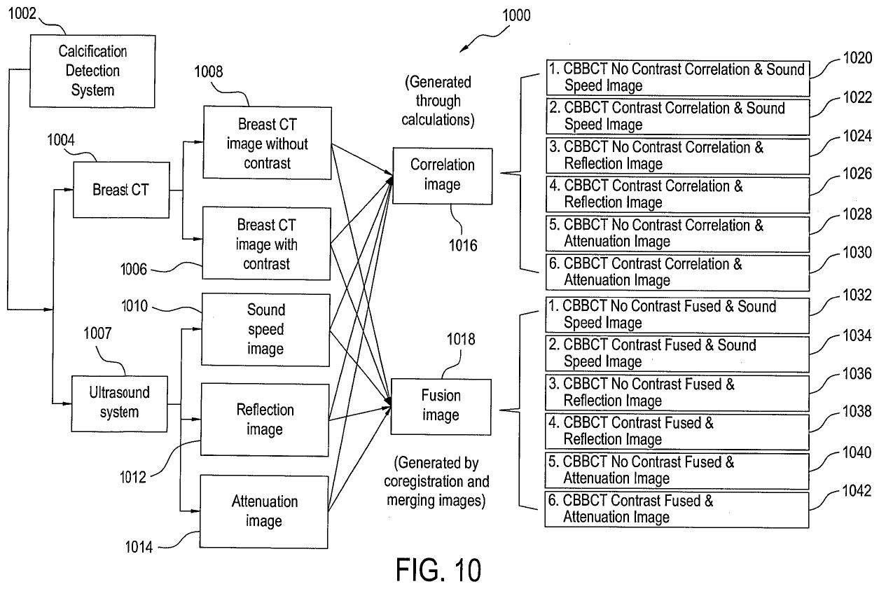

FIG. 10 shows, in schematic block diagram form, exemplary aspects of processing systems and methods according to principles of the invention 1000, including 12 exemplary operational modalities.

Consistent with the totality of the description above, a CBBCT system 1002, including ultrasonic hybrid imaging features according to the present invention, is configured to prepare an enhanced tomographic data set for Cone Beam Breast Computed Tomography of a subject breast. The CBBCT image produced by system 1002 is enhanced by image processing of one or more tomographic images with one or more ultrasound images acquired by the hybrid CBBCT system.

Accordingly, CBBCT system 1002 is configured and operated to acquire one or more CBBCT image data sets 1004. Depending on a selected mode of operation, the one or more acquired CBBCT image data sets 1004 is acquired with the use of an injected contrast agent 1006, and/or the one or more acquired CBBCT image data sets 1004 is acquired without the use of an injected contrast agent 1008.

The CBBCT system 1002 is further configured and operated to acquire one or more ultrasound image data sets 1007. Depending on a selected mode of operation, the one or more acquired ultrasound image data sets 1007 is acquired as a Sound Speed Image 1010; and/or the one or more acquired ultrasound image data sets 1007 is acquired as a Reflection Image 1012; and/or the one or more acquired ultrasound image data sets 1007 is acquired as an Attenuation Image 1014.

A processor of the CBBCT system 1002 is configured and operated to receive the respective data sets acquired as indicated above, and to combine them through tomographic calculations to produce a correlation image 1016. In alternate modes of operation, CBBCT system, 1002 is further configured and operated to receive the respective data sets acquired as indicated above, and to combine them through co-registration and merging images to produce a fusion image 1018.

Accordingly, where a correlation image 1016 is produced, such a correlation image will be produced in one or more of the following modalities:
1. CBBCT image without contrast correlated with a Sound Speed image 1020.
2. CBBCT image with contrast correlated with a Sound Speed image 1022.
3. CBBCT image without contrast correlated with Reflection image 1024.
4. CBBCT image with contrast correlated with a Reflection image 1026.
5. CBBCT image without contrast correlated with an Attenuation image 1028.
6. CBBCT image with contrast correlated with an Attenuation image 1030.

Accordingly, where a fusion image 1018 is produced, such a fusion image will be produced in one or more of the following modalities:
1. CBBCT image without contrast fused with a Sound Speed image 1032.
2. CBBCT image with contrast fused with a Sound Speed image 1034.
3. CBBCT image without contrast fused with a Reflection image 1036.
4. CBBCT image with contrast fused with a Reflection image 1038.
5. CBBCT image without contrast fused with an Attenuation image 1040.
6. CBBCT image with contrast fused with an Attenuation image 1042.

In view of the disclosure associated with FIG. 10 and, more broadly, in view of the totality of the present disclosure, one of skill in the art will readily appreciate that the image acquisition and processing methods and modalities presented herewith are merely exemplary of a much larger set of methods and modalities. These novel methods and modalities, while surprising and effective, become clear to the practitioner of ordinary skill in the art once having been exposed to the present disclosure. Accordingly, while an extensive listing of all possible methods and modalities exceeds the reasonable scope of the present document, it should be understood that this document is intended to disclose the entirety of the methods, systems and apparatus, structure, methods and results that would become apparent to the skilled practitioner in possession of the present disclosure.

In certain embodiments a hybrid imaging system includes a structural member and a CBBCT x-ray subsystem coupled to and supported by the structural member. An ultrasound subsystem is coupled to and supported by the structural member. A processor is signalingly coupled to the CBBCT x-ray subsystem and to the ultrasound subsystem, the processor being adapted to receive signals from the CBBCT x-ray system and the ultrasound subsystem respectively and to produce an x-ray-ultrasound hybrid CBBCT image.

In certain embodiments the CBBCT x-ray subsystem includes a rotating gantry, the rotating gantry being supported by the structural member through a bearing.

In certain embodiments the ultrasound subsystem is supported by a rotating gantry.

In certain embodiments the ultrasound subsystem includes a rotary apparatus, the rotary apparatus being supported by the structural member for rotation independent of the gantry.

In certain embodiments the ultrasound subsystem includes a rotary apparatus and a base portion, with the base portion being mounted on and supported by the rotary apparatus. The ultrasound subsystem includes a translation portion and an ultrasound transducer assembly.

In some embodiments the base portion includes a first circumferential actuator, a second radial actuator, and a third vertical actuator.

In some embodiments the first ultrasound assembly includes an ultrasound transducer while in certain embodiments the first ultrasound assembly includes a feedback sensor.

In some embodiments the first ultrasound assembly includes a lubricant port.

In certain embodiments a method of producing a hybrid breast image with an integrated breast imaging system includes operatively positioning a patient breast in relation to the integrated breast imaging system, imaging the patient breast with an x-ray CBBCT subsystem of the breast imaging system while maintaining the patient breast substantially immobile with respect to the integrated breast imaging system, imaging the patient breast with an ultrasound subsystem of the breast imaging system while maintaining the patient breast substantially immobile with respect to the integrated breast imaging system and maintaining the patient breast substantially immobile with respect to the integrated breast imaging system during an entire time duration between the x-ray CBBCT imaging and the ultrasound CBBCT imaging.

In some embodiments a method of producing a hybrid breast image with an integrated breast imaging system includes receiving x-ray CBBCT imaging data from the x-ray CBBCT subsystem at an image processing system, receiving ultrasound imaging data from the ultrasound subsystem at the image processing system and producing a hybrid image data set with the image processing system. The hybrid image data set includes image data hybridized from the x-ray CBBCT imaging data and the ultrasound imaging data.

In certain embodiments a method of producing a hybrid breast image with an integrated breast imaging system includes disposing a patient on a patient support table.

In certain embodiments the ultrasound subsystem comprises a reflective ultrasound system whereas in some embodiments the ultrasound subsystem comprises a transmission ultrasound system.

In still further embodiments the ultrasound subsystem comprises an ultrasound computed tomography system. In some embodiment the ultrasound subsystem comprises an automated ultrasound system.

In certain embodiments an automated ultrasound system comprises a pivotal apparatus, a support column and an ultrasound transducer portion.

In certain embodiments an ultrasound transducer portion of the ultrasound imaging subsystem moves through a circumferential spiral-helical motion across a surface region of a breast. In certain embodiments an ultrasound transducer portion of the ultrasound imaging subsystem moves through a radial/longitudinal motion across a surface region of a breast.

In certain embodiments the method of producing a hybrid breast image with an integrated breast imaging system includes receiving x-ray CBBCT imaging data from an x-ray CBBCT subsystem at an image processing system and receiving ultrasound imaging data from an ultrasound subsystem at an image processing system. After receiving the x-ray CBBCT imaging data and receiving the ultrasound imaging data at the image processing system, receiving further x-ray CBBCT imaging data at the image processing system. After receiving the x-ray CBBCT imaging data the x-ray CBBCT system and receiving the ultrasound imaging data from the ultrasound subsystem at the image processing system, receiving further ultrasound imaging data from the ultrasound subsystem at the image processing system and producing a hybrid image data set. The hybrid image data set includes image data hybridized from the x-ray CBBCT imaging data and the ultrasound imaging data.

While the exemplary embodiments described above have been chosen primarily from the field of apparatus, and corresponding systems and methods, for secondary imaging during the operation of a CBBCT imaging system, including hybrid x-ray ultrasonic breast imaging, one of skill in the art will appreciate that the principles of the invention are equally well applied, and that the benefits of the present invention are equally well realized in a wide variety of other imaging technologies, for example, imaging of other body parts and imaging of other subjects such as industrial and technological products.

Further, while the invention has been described in detail in connection with the presently preferred embodiments, it should be readily understood that the invention is not limited to such disclosed embodiments. Rather, the invention can be modified to incorporate any number of variations, alterations, substitutions, or equivalent arrangements not heretofore described, but which are commensurate with the spirit and scope of the invention. Accordingly, the invention is not to be seen as limited by the foregoing description, but is only limited by the scope of the appended claims.

1. A hybrid imaging system comprising:
a structural member;
a CBBCT x-ray subsystem coupled to and supported by said structural member;
an ultrasound subsystem coupled to and supported by said structural member;
a processor, said processor signalingly coupled to said CBBCT x-ray subsystem and to said ultrasound subsystem, said processor being adapted to receive signals from said CBBCT x-ray system and said ultrasound subsystem respectively and to produce an x-ray-ultrasound hybrid CBBCT image;
wherein said CBBCT x-ray subsystem includes a rotating gantry, said rotating gantry being supported by said structural member through a bearing; and
wherein said ultrasound subsystem includes a rotary apparatus, said rotary apparatus being supported by said structural member for rotation independent of said gantry.

2. A hybrid imaging system as defined in claim 1 wherein said ultrasound subsystem is supported by said rotating gantry.

3. A hybrid imaging system comprising:
a structural member;
a CBBCT x-ray subsystem coupled to and supported by said structural member; an ultrasound subsystem coupled to and supported by said structural member; a processor, said processor signalingly coupled to said CBBCT x-ray subsystem and to said ultrasound subsystem, said processor being adapted to receive signals from said CBBCT x-ray system and said ultrasound subsystem respectively and to produce an x-ray-ultrasound hybrid CBBCT image;
wherein said ultrasound subsystem comprises:
a rotary apparatus;
a base portion, said base portion being mounted on and supported by said rotary apparatus;
a translation portion; and
an ultrasound transducer assembly.

4. A hybrid imaging system as defined in claim 3 wherein said ultrasound subsystem is supported by said rotating gantry.

5. A hybrid imaging system as defined in claim 5 wherein said base portion includes a first circumferential actuator, a second radial actuator, and a third vertical actuator.

6. A hybrid imaging system as defined in claim 5 wherein said first ultrasound assembly includes an ultrasound transducer.

7. A hybrid imaging system as defined in claim 5 wherein said first ultrasound assembly includes a feedback sensor.

8. A hybrid imaging system as defined in claim 5 wherein said first ultrasound assembly includes a lubricant port.

9. A method of producing a hybrid breast image with an integrated breast imaging system comprising:
operatively positioning a patient breast in relation to said integrated breast imaging system;
imaging said patient breast with an x-ray CBBCT subsystem of said breast imaging system while maintaining said patient breast substantially immobile with respect to said integrated breast imaging system;
imaging said patient breast with an ultrasound subsystem of said breast imaging system while maintaining said patient breast substantially immobile with respect to said integrated breast imaging system; and
maintaining said patient breast substantially immobile with respect to said integrated breast imaging system during an entire time duration between said x-ray CBBCT imaging and said ultrasound CBBCT imaging.

10. A method of producing a hybrid breast image with an integrated breast imaging system as defined in claim 9, further comprising:
receiving x-ray CBBCT imaging data from said x-ray CBBCT subsystem at an image processing system;
receiving ultrasound imaging data from said ultrasound subsystem at said image processing system; and
producing a hybrid image data set with said image processing system, said hybrid image data set comprising image data hybridized from said x-ray CBBCT imaging data and said ultrasound imaging data.

11. A method of producing a hybrid breast image with an integrated breast imaging system as defined in claim 9, wherein said ultrasound subsystem comprises a reflective ultrasound system.

12. A method of producing a hybrid breast image with an integrated breast imaging system as defined in claim 9, wherein said ultrasound subsystem comprises a transmission ultrasound system.

13. A method of producing a hybrid breast image with an integrated breast imaging system as defined in claim 9, wherein said ultrasound subsystem comprises an ultrasound computed tomography system.

14. A method of producing a hybrid breast image with an integrated breast imaging system as defined in claim 9, wherein said ultrasound subsystem comprises an automated ultrasound system.

15. A method of producing a hybrid breast image with an integrated breast imaging system as defined in claim 14, wherein said automated ultrasound system comprises a pivotal apparatus, a support column and an ultrasound transducer portion.

16. A method of producing a hybrid breast image with an integrated breast imaging system as defined in claim 14, further comprising moving an ultrasound transducer portion of said ultrasound imaging subsystem through a circumferential spiral-helical motion across a surface region of said breast.

17. A method of producing a hybrid breast image with an integrated breast imaging system as defined in claim 14, further comprising moving an ultrasound transducer portion of said ultrasound imaging subsystem through a radial/longitudinal motion across a surface region of said breast.
